(19) United States
(12) Patent Application Publication
(10) Pub. No.: US 2024/0366606 A1
(43) Pub. Date: Nov. 7, 2024

(54) MRGPRX2 ANTAGONISTS AND USES THEREOF

(71) Applicants: The Regents of the University of California, Oakland, CA (US); The University of North Carolina at Chapel Hill, Chapel Hill, NC (US)

(72) Inventors: Brian K. Shoichet, Kentfield, CA (US); Isha Singh, Redwood City, CA (US); Bryan L. Roth, Durham, NC (US); Can Cao, Chapel Hill, NC (US); Hye Jin Kang, Chapel Hill, NC (US)

(21) Appl. No.: 18/688,708

(22) PCT Filed: Sep. 7, 2022
(86) PCT No.: PCT/US22/76075

§ 371 Date: Mar. 1, 2024

Related U.S. Application Data
(60) Provisional application No. 63/241,849, filed on Sep. 8, 2021.

(51) Int. Cl. A61K 31/517

(52) U.S. Cl. CPC A61K 31/517

(57) ABSTRACT

Described herein, inter alia, are MRGPRX2 antagonists and uses thereof.

### CROSS-REFERENCES TO RELATED APPLICATIONS

This application claims the benefit of U.S. Provisional Application No. 63/241,849, filed Sep. 8, 2021, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. U24 DK116195 and R35 GM122481 awarded by The National Institutes of Health. The government has certain rights in the invention.

### REFERENCE TO AN ELECTRONIC SEQUENCE LISTING

The contents of the electronic sequence listing (048536-720001WO_ST26.xml; Size: 2,054 bytes; and Date of Creation: Sep. 7, 2022) is hereby incorporated by reference in its entirety.

### BACKGROUND

The sensation of itch, or pruritus, can be triggered by many environmental insults including insect bites and parasites, skin diseases such as eczema, liver and kidney diseases, and hypersensitivity reactions to commonly prescribed medications. The itch sensation has both neuronal and non-neuronal components; in the latter histamine released from mast cells is prominent. Several transmitters and molecular targets have been implicated in sensing and mediating the itch response. Among the transmitters these include histamine, interleukin, various peptides, while among the molecular targets G protein-coupled receptors (GPCRs), cytokine receptors, and ion channels are involved. Recently, Mas-related G protein-coupled receptors (MRGPRs) have been identified as pruritogenic receptors. Disclosed herein, inter alia, are solutions to these and other problems in the art.

### BRIEF SUMMARY

In an aspect is provided a compound, or a pharmaceutically acceptable salt thereof, having the formula:

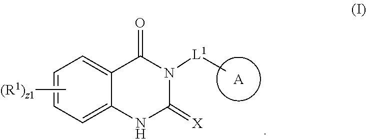

X is S or O.

Ring A is substituted or unsubstituted cycloalkyl, substituted or unsubstituted heterocycloalkyl, substituted or unsubstituted aryl, or substituted or unsubstituted heteroaryl.

L¹ is substituted or unsubstituted alkylene.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl.

X¹ is independently —F, —Cl, —Br, or —I.

The symbol n1 is an integer from 0 to 4. The symbols m1 and v1 are independently 1 or 2. The symbol z1 is an integer from 0 to 4.

In an aspect is provided a pharmaceutical composition including a compound described herein, or a pharmaceutically acceptable salt thereof, and a pharmaceutically acceptable excipient.

In an aspect is provided a method of treating pruritus in a subject in need thereof, the method including administering to the subject in need thereof a therapeutically effective amount of a compound described herein, or a pharmaceutically acceptable salt thereof.

In an aspect is provided a method of treating mast cell-mediated hypersensitivity in a subject in need thereof, the method including administering to the subject in need thereof a therapeutically effective amount of a compound described herein, or a pharmaceutically acceptable salt thereof.

In an aspect is provided a method of treating pain in a subject in need thereof, the method including administering to the subject in need thereof a therapeutically effective amount of a compound described herein, or a pharmaceutically acceptable salt thereof.

In an aspect is provided a method of treating an inflammatory disease in a subject in need thereof, the method including administering to the subject in need thereof a therapeutically effective amount of a compound described herein, or a pharmaceutically acceptable salt thereof.

In an aspect is provided a method of treating an autoimmune disease in a subject in need thereof, the method including administering to the subject in need thereof a therapeutically effective amount of a compound described herein, or a pharmaceutically acceptable salt thereof.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of decreasing the level of activity of MRGPRX2 in a cell, said method comprising contacting the cell with an effective of a compound described herein, or a pharmaceutically acceptable salt thereof.

### BRIEF DESCRIPTION OF THE DRAWINGS

FIGS. 1A-1J. CryoEM structures of MRGPRX2 complexes. FIGS. 1A-ID: Cartoon representations of MRGPRX2-Gq-cortistatin-14 complex (FIG. 1A), MRGPRX2-Gq-(R)-ZINC-3573 complex (FIG. 1B), MRGPRX2-Gi1-cortistatin-14 complex (FIG. 1C) and the MRGPRX2-Gi1-(R)-ZINC-3573 complex (FIG. 1D). FIG. 1E: Electrostatic surface representation of the MRGPRX2 extracellular pocket with cortistatin-14 shown as sticks. The cross-section image shows a nice fit of Lys3 of cortistatin-14 (spheres) to sub-pocket1. Electrostatic potential surface was calculated using the APBS plugin in PyMOL. FIG. 1F: Binding pocket of Cortistatin-14. Key residues of MRGPRX2 interacting with the Lys3 of corstitatin-14 were shown as sticks. Hydrogen bonds are shown as dashed lines. FIG. 1G: Key residues involved in (R)-ZINC-3573 binding in MRGPRX2. Charge interaction is shown as dashed lines. FIG. 1H: Electrostatic surface representation of the MRGPRX2 extracellular pocket with (R)-ZINC-3573 shown as sticks. FIG. 1I: Alanine substitution of MRGPRX2 residues interact with the Lys3 of cortistatin-14 significantly reduced cortistatin-14 stimulated BRET2 Gq activation. HEK293T cells were used. Data represent mean SEM of n=3 biological replicates. FIG. 1J: BRET2 validation of the (R)-ZINC-3573 binding pocket. HEK293T cells were used for the functional studies. Data represent mean SEM of n=3 biological replicates.

FIGS. 2A-2E. G protein coupling of MRGPRX2. FIG. 2A: The α5 helix of Gq engages the cytoplasmic core of MRGPRX2 in a way distinct from Gi1. The relative displacement of Gq with respect to Gi1 is indicated by arrow. FIGS. 2B-2C: The detailed interactions of ICL3 (FIG. 2B) and ICL2 of MRGPRX2 with Gi1 (FIG. 2C). The hydrogen bonds are highlighted as dashed lines. FIG. 2D: Different engagement mode of the αN helix of Gq and Gi upon coupling to MRGPRX2. The relative displacements of Gq with respect to Gi1 are indicated by arrows. FIG. 2E: The detail interaction of ICL2 of MRPGRX2 with Gq. Hydrogen bonds are highlighted as dashed lines.

FIGS. 3A-3G. Discovery of MRGPGRX2 selective inverse agonists. FIG. 3A: Overview of the analog optimization toward compound C9 and C9-6. FIG. 3B: C9 and C9-6 show improved antagonist activity for MRGPRX2 when compared to the parent compound '1592. C7 is shown as an inactive control. EC₈₀ (R)-ZINC-3573 concentration (3 μM) was added in the antagonist mode FLIPR Ca²⁺ assay using a tetracycline-inducible MRGPRX2 stable cell line. Data represent mean±SEM of n=3 biological replicates. FIG. 3C: Compound C9 and C9-6 inhibit the basal recruitment of Gq by MRGPRX2 and display inverse agonist activities. HEK293T cells were used. Data represent mean±SEM of n=3 biological replicates. FIG. 3D: C9 and C9-6 inhibit substance P stimulated MRGPRX2 activation. Tetracycline-inducible MRGPRX2 stable cell lines were used in FLIPR assay with EC₈₀ substance P concentration. Data represent mean±SEM of n=3 biological replicates. FIGS. 3E-3F: C9 and C9-6 display no antagonist activity towards NK1R (FIG. 3E) and MRGPRX4 (FIG. 3F). Tetracycline-inducible NK1R and MRGPRX4 stable cell lines were used in FLIPR assay. Agonist concentrations in the antagonist assay were shown in the graph title. C7 is used as a negative control. Data represent mean±SEM of n=3 biological replicates. FIG. 3G: Compound C9 inhibit MRGPRX2 mediated LAD2 human mast activation. Data represent mean±SEM. Samples were run in quadruplicate and each compound was assayed in at least two independent experiments.

FIG. 4. MRGPRX2 transducerome screening using TRUPATH. MRGPRX2 effectively couples to 14 distinct G proteins upon stimulation of agonists (R)-ZINC-3573 and cortistatin-14 (C-14) and HEK293T cells were used. Net BRET values of MRGPRX2 together with positive controls of either neurotensin-1 receptor (NT1R, agonist NT1-13) or β2AR (agonist isoproterenol) are shown in each panel. Data represent mean±SEM of n=3 biological replicates.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom.

Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

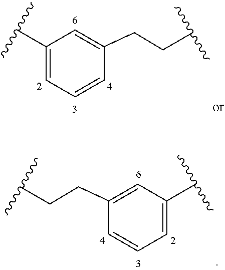 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3.

Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; RL^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{W} group is phenyl substituted by R^{WW.1}, which is methyl, the methyl group may be further substituted to form groups including but not limited to:

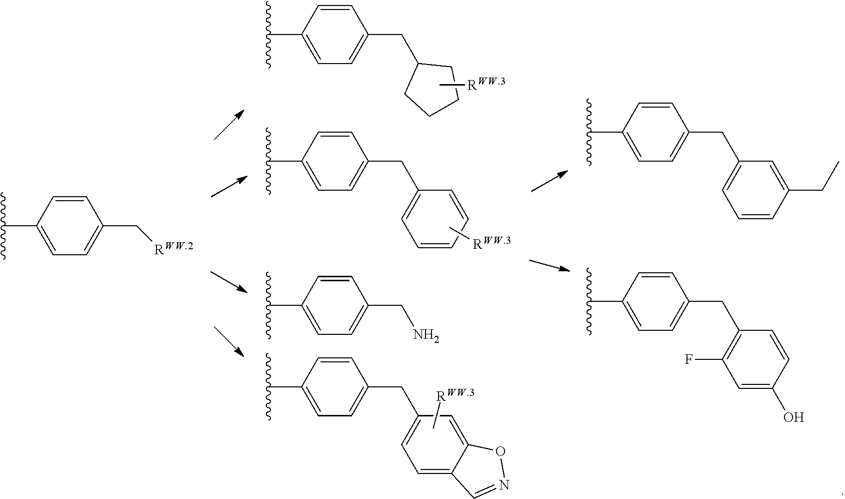

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NH₄NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NH₄NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted.

Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW} is R^{100A.1} R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}_substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{L}WW. ³-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}. —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.).

R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{WW.1}-substituted or unsubstituted cycloalkylene (e.g., C₃-C₈, C₃-C₆, C₄-C₆, or C₅-C₆), R^{WW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms.

In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is pruritus. In embodiments, the disease is mast cell-mediated hypersensitivity. In embodiments, the disease is pain. In embodiments, the disease is an inflammatory disease. In embodiments, the disease is an autoimmune disease. In embodiments, the disease is cancer.

The term “pruritus” as used herein refers to a disease or condition characterized by aberrant itch (e.g., an increased level of itch compared to a control such as a healthy person not suffering from a disease).

The term “chronic prurigo” as used herein refers to a disease or condition characterized by pruritus that lasts for an extended period (e.g. at least 6 weeks). Chronic prurigo may include, a history or signs of repeated scratching, and/or multiple localized or generalized pruritic skin lesions (e.g. whitish or pinkish papules, nodules or plaques). In embodiments, chronic prurigo is associated with MRGPRX2 expression or activity (e.g. Kolkhir et al. J Allergy Clin Immunol 2022, 149(6):1998-2009, the disclosure of which is hereby incorporated by reference in its entirety for all purposes).

The term “prurigo nodularis” as used herein refers to a disease or condition characterized by pruritus which causes nodules on the skin. In embodiments the nodules are hard, pruritic lumps on the skin. In embodiments, the prurigo nodularis is caused by atopic dermatitis. In embodiments, the prurigo nodularis is caused by contact dermatitis. In embodiments, the prurigo nodularis is caused by diabetes. In embodiments, the prurigo nodularis is caused by kidney disease. In embodiments, the prurigo nodularis is caused by hepatitis C infection. In embodiments, the prurigo nodularis is caused by HIV infection or AIDS. In embodiments, the prurigo nodularis is caused by cancer. In embodiments, the prurigo nodularis is caused by lymphoma. In embodiments, the prurigo nodularis is caused by neuropsychiatric disease or conditions (e.g. anxiety, depression, etc.).

The term “urticaria” as used herein refers to a disease or condition characterized by pruritic raised skin areas (e.g. welts or hives on the skin of a person). In embodiments, the urticaria is caused by a food allergy. In embodiments, the urticaria is caused by prescription medication. In embodiments, the urticaria is caused by an environmental allergy. In embodiments, the urticaria is caused by a bacterial infection. In embodiments, the urticaria is caused by a viral infection. In embodiments, the urticaria is caused by an environmental stimulus (e.g. pressure, temperature, sun exposure, etc.). In embodiments, the urticaria is caused by a surgical procedure. In embodiments, the urticaria is caused by cancer. In embodiments, the urticaria is caused by thyroid disease. In embodiments, chronic urticaria is a disease or condition in which the urticaria last more than six weeks and recurring over months or years. In embodiments, chronic spontaneous urticaria is a disease or condition characterized by chronic urticaria independent of any exogenous stimulus.

The term “rosacea” is used herein according to its plain and ordinary meaning and refers to an inflammatory skin condition characterized by redness and/or rash (e.g., red or pus-filled bumps and pimples). In embodiments, the rosacea is caused by inflammation. In embodiments, the rosacea is caused by an autoimmune disease or condition. In embodiments, the rosacea is caused by an environmental stressor (e.g ultraviolet light, temperature extremes, etc.). In embodiments, the rosacea is caused by a microbe (e.g. microbiota on the skin of a person).

The term “neurogenic inflammation” is used herein according to its plain and ordinary meaning and refers to inflammation initiated and/or mediated by neurons in the peripheral nervous system (e.g. through their release of neuropeptides such as calcitonin gene related peptide (CGRP), substance P, etc.). In embodiments, the neurogenic inflammation causes migraine. In embodiments, the neurogenic inflammation causes psoriasis. In embodiments, the neurogenic inflammation causes asthma. In embodiments, the neurogenic inflammation causes fibromyalgia. In embodiments, the neurogenic inflammation causes eczema. In embodiments, the neurogenic inflammation causes rosacea.

The term “migraine” as used herein refers to a disease or condition characterized by recurring headaches that cause moderate to severe pain. In embodiments, migraine is associated with neurogenic inflammation or MRGPRX2 expression or activity (e.g. Lukas et al. Curr Med Chem 2017, 24:3649-65; Tuka et al. Cephalagia 2013, 33:1085-95; Fusayasu et al. Pain 2007, 128:209-14; Ogasawara et al. Cells 2021, 10:2906; Ottosson et al. Cephalagia 1997, 17:166-74; Okragly et al. Cephalagia 2018, 38:1564-74; Amin et al. Brain 2014, 137:779-94; Schytz et al. Brain 2009, 132:16-25; and Sicuteri et al. Headache 1963, 3(3):86-92, the disclosure of each is hereby incorporated by reference in their entirety for all purposes).

The term “neuropathic pain” is used herein according to its plain and ordinary meaning and refers to pain caused by a damaged or not fully-functioning nervous system. In embodiments, the neuropathic pain is caused by diabetes. In embodiments, the neuropathic pain is caused by alcoholism. In embodiments, the neuropathic pain is caused by HIV infection or AIDS. In embodiments, the neuropathic pain is caused by a CNS disorder such as Parkinson's disease or multiple sclerosis. In embodiments, the neuropathic pain is caused by shingles. In embodiments, the neuropathic pain is caused by a bacterial or viral infection. In embodiments, the neuropathic pain is caused by radiation therapy, chemotherapy, amputation, spinal nerve compression, trauma or surgeries resulting in nerve damage, nerve compression of infiltration by tumors. In embodiments, neuropathic pain is caused by a lesion or disease of the somatosensory nervous system.

The term“peripheral neuropathy” is used herein according to its plain and ordinary meaning and refers to pain, numbness and/or weakness caused by damaged or not fully-functioning nerves located outside of the brain and spinal cord.

The term “mast cell-mediated hypersensitivity” or “Type I hypersensitivity” as used herein refers to a hypersensitive immune reaction (e.g., allergic response) and involves mast cell degranulation and release of inflammatory mediators (e.g., histamine, prostaglandins, leukotrienes, kinins, serotonin, heparin, and serine proteases).

The term “perioperative anaphylaxis” is used herein according to its plain and ordinary meaning and refers to anaphylaxis that occurs during the perioperative period. In embodiments, perioperative anaphylaxis is a hypersensitivity that is IgE-mediated. In embodiments, the perioperative anaphylaxis is a life-threatening immediate hypersensitivity condition.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient).

A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶y, ⁹⁰Y ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., pruritus, mast cell-mediated hypersensitivity, pain, inflammatory disease, autoimmune disease, or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., pruritus, mast cell-mediated hypersensitivity, pain, inflammatory disease, autoimmune disease, or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to E164 of MRGPRX2 when the selected residue occupies the same essential spatial or other structural relationship as E164 of MRGPRX2. In some embodiments, where a selected protein is aligned for maximum homology with MRGPRX2, the position in the aligned selected protein aligning with E164 is said to correspond to E164. Instead of a primary sequence alignment, a three dimensional structural alignment can also be used, e.g., where the structure of the selected protein is aligned for maximum correspondence with MRGPRX2 and the overall structures compared. In this case, an amino acid that occupies the same essential position as E164 in the structural model is said to correspond to the E164 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “Mas-related G protein-coupled receptor” or “MRGPR” refers to one or more of the family of G protein-coupled receptors that play a role in neuro-immune biological processes (e.g., itch, pain, and inflammation). In embodiments, MRGPR includes MRGPRX1, MRGPRX2, MRGPRX3, and MRGPRX4.

The term “Mas-related G protein-coupled receptor member X2” or “MRGPRX2” refers to a protein (including homologs, isoforms, and functional fragments thereof) that mediates allergic reactions characterized by histamine release. The term includes any recombinant or naturally-occurring form of MRGPRX2 variants thereof that maintain MRGPRX2 activity (e.g., within at least 30%, 40%, 50%, 60%, 70%, 80%, 90%, 95%, or 100% activity compared to wildtype MRGPRX2). In embodiments, the MRGPRX2 protein encoded by the MRGPRX2 gene has the amino acid sequence set forth in or corresponding to Entrez 117194, UniProt Q96LB1, RefSeq (protein) NP_001290544.1, or RefSeq (protein) NP_473371.1. In embodiments, the MRGPRX2 gene has the nucleic acid sequence set forth in RefSeq (mRNA) NM_001303615.1 or RefSeq (mRNA) NM_054030.3. In embodiments, the amino acid sequence or nucleic acid sequence is the sequence known at the time of filing of the present application. In embodiments, the MRGPRX2 protein has the amino acid sequence:

(SEQ ID NO: 1)
MDPTTPAWGTESTTVNGNDQALLLLCGKETLIPVFLILFIALVGLV
GNGFVLWLLGFRMRRNAFSVYVLSLAGADFLFLCFQIINCLVYLS
NFFCSISINFPSFFTTVMTCAYLAGLSMLSTVSTERCLSVLWPIW
YRCRRPRHLSAVVCVLLWALSLLLSILEGKFCGFLFSDGDSGWCQ
TFDFITAAWLIFLFMVLCGSSLALLVRILCGSRGLPLTRLYLTIL
LTVLVFLLCGLPFGIQWFLILWIWKDSDVLFCHIHPVSVVLSSLN
SSANPIIYFFVGSFRKQWRLQQPILKLALQRALQDIAEVDHSEGC
FRQGTPEMSRSSLV.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward MRGPRX2 would preferentially inhibit MRGPRX2 over other Mas-related G protein-coupled receptors). In embodiments, an “MRGPRX2-selective compound” refers to a compound (e.g., compound described herein) having selectivity towards MRGPRX2. In embodiments, the compound (e.g., compound described herein) is about 5-fold, 10-fold, 20-fold, 30-fold, 40-fold, 50-fold, or about 100-fold more selective for MRGPRX2 over one or more of MRGPRX1, MRGPRX3, or MRGPRX4. In embodiments, the compound (e.g., compound described herein) is at least 5-fold, 10-fold, 20-fold, 30-fold, 40-fold, 50-fold, or at least 100-fold more selective for MRGPRX2 over one or more of MRGPRX1, MRGPRX3, or MRGPRX4.

### II. Compounds

In an aspect is provided a compound, or a pharmaceutically acceptable salt thereof, having the formula:

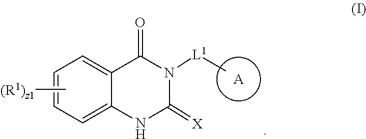

X is S or O.

Ring A is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L¹ is substituted or unsubstituted alkylene (e.g., C₁-C₈, C₁-C₆, C₁-C₄, or C₁-C₂).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B}substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹ is independently —F, —Cl, —Br, or —I.

The symbol n1 is an integer from 0 to 4.

The symbols m1 and v1 are independently 1 or 2.

The symbol z1 is an integer from 0 to 4.

In embodiments, the compound has the formula:

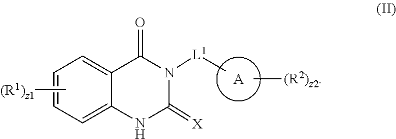

X, L¹, R¹, and z1 are as described herein, including in embodiments.

In embodiments, Ring A is cycloalkyl, heterocycloalkyl, aryl, or heteroaryl.

R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X² is independently —F, —Cl, —Br, or —I.

The symbol n2 is an integer from 0 to 4.

The symbols m2 and v2 are independently 1 or 2.

The symbol z2 is an integer from 0 to 11.

In embodiments, X is S. In embodiments, X is 0.

In embodiments, a substituted L¹ (e.g., substituted alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is unsubstituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted isobutylene. In embodiments, L¹ is unsubstituted tert-butylene.

In embodiments, a substituted Ring A (e.g., substituted cycloalkyl, substituted heterocycloalkyl, substituted aryl, and/or substituted heteroaryl) is substituted with at least one substituent group, size-limited substituent group, or lower substituent group; wherein if the substituted Ring A is substituted with a plurality of groups selected from substituent groups, size-limited substituent groups, and lower substituent groups; each substituent group, size-limited substituent group, and/or lower substituent group may optionally be different. In embodiments, when Ring A is substituted, it is substituted with at least one substituent group. In embodiments, when Ring A is substituted, it is substituted with at least one size-limited substituent group. In embodiments, when Ring A is substituted, it is substituted with at least one lower substituent group.

In embodiments, Ring A is substituted or unsubstituted cycloalkyl. In embodiments, Ring A is substituted or unsubstituted heterocycloalkyl. In embodiments, Ring A is substituted or unsubstituted aryl. In embodiments, Ring A is substituted or unsubstituted phenyl. In embodiments, Ring A is substituted or unsubstituted heteroaryl. In embodiments, Ring A is substituted or unsubstituted 5 to 6 membered heteroaryl. In embodiments, Ring A is substituted or unsubstituted pyridyl. In embodiments, Ring A is substituted or unsubstituted 2-pyridyl. In embodiments, Ring A is substituted or unsubstituted 3-pyridyl. In embodiments, Ring A is substituted or unsubstituted 4-pyridyl. In embodiments, Ring A is substituted or unsubstituted pyrazinyl. In embodiments, Ring A is substituted or unsubstituted pyrimidinyl. In embodiments, Ring A is substituted or unsubstituted pyridazinyl. In embodiments, Ring A is substituted or unsubstituted oxazolyl. In embodiments, Ring A is substituted or unsubstituted isoxazolyl. In embodiments, Ring A is substituted or unsubstituted oxadiazolyl. In embodiments, Ring A is substituted or unsubstituted imidazolyl. In embodiments, Ring A is substituted or unsubstituted thiazolyl.

In embodiments, Ring A is substituted or unsubstituted thiadiazolyl.

In embodiments, Ring A is cycloalkyl. In embodiments, Ring A is heterocycloalkyl. In embodiments, Ring A is aryl. In embodiments, Ring A is phenyl. In embodiments, Ring A is heteroaryl. In embodiments, Ring A is 5 to 6 membered heteroaryl. In embodiments, Ring A is pyridyl. In embodiments, Ring A is 2-pyridyl. In embodiments, Ring A is 3-pyridyl. In embodiments, Ring A is 4-pyridyl. In embodiments, Ring A is pyrazinyl. In embodiments, Ring A is pyrimidinyl. In embodiments, Ring A is pyridazinyl. In embodiments, Ring A is oxazolyl. In embodiments, Ring A is isoxazolyl. In embodiments, Ring A is oxadiazolyl. In embodiments, Ring A is imidazolyl. In embodiments, Ring A is thiazolyl. In embodiments, Ring A is thiadiazolyl.

In embodiments,

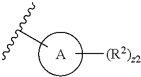

is

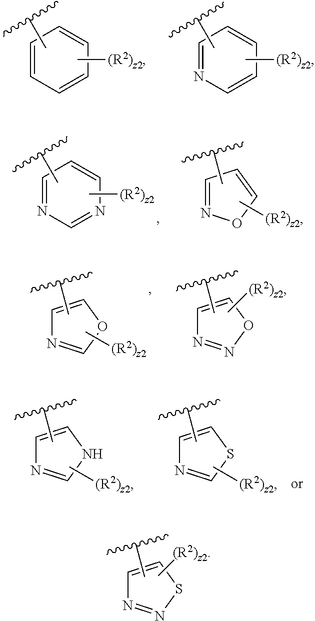

R² and z2 are as described herein, including in embodiments.

In embodiments,

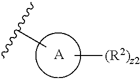

is

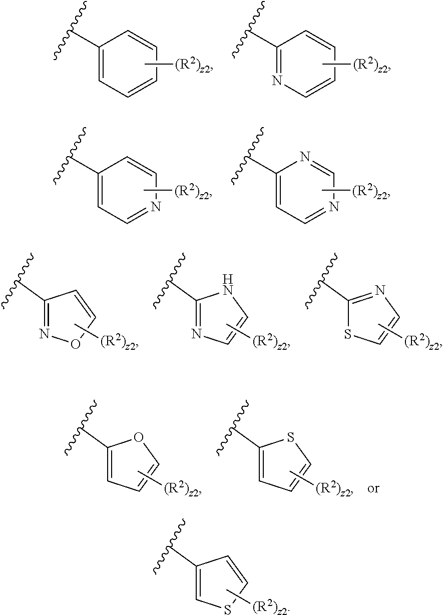

R² and z2 are as described herein, including in embodiments.

In embodiments,

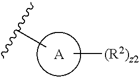

is

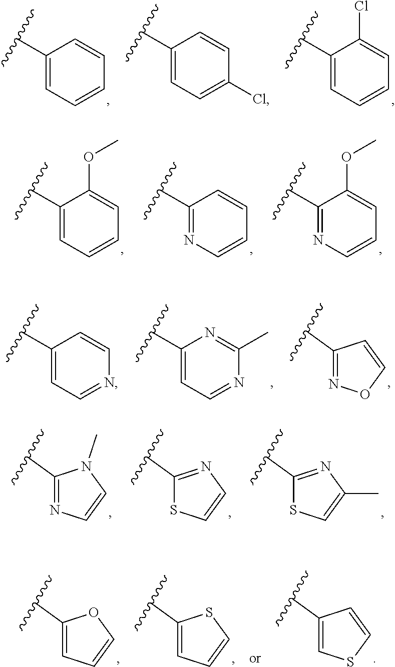

In embodiments, the compound has the formula:

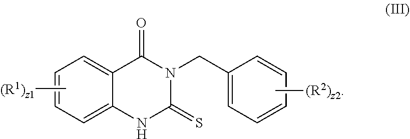

R¹, z1, R², and z2 are as described herein, including in embodiments.

In embodiments, the compound has the formula:

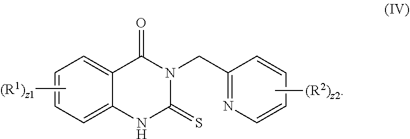

R¹, z1, R², and z2 are as described herein, including in embodiments.

In embodiments, the compound has the formula:

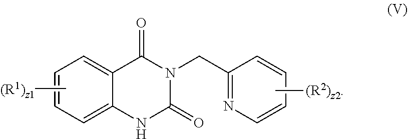

R¹, z1, R², and z2 are as described herein, including in embodiments.

In embodiments, the compound has the formula:

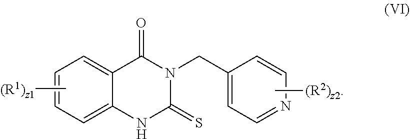

R¹, z1, R², and z2 are as described herein, including in embodiments.

In embodiments, the compound has the formula:

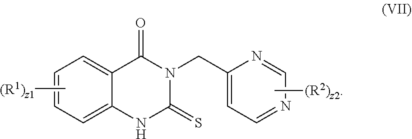

R¹, z1, R², and z2 are as described herein, including in embodiments.

In embodiments, the compound has the formula:

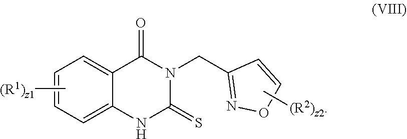

R¹, z1, R², and z2 are as described herein, including in embodiments.

In embodiments, the compound has the formula:

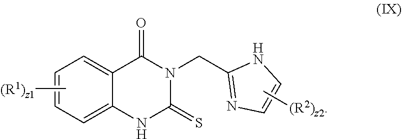

R¹, z1, R², and z2 are as described herein, including in embodiments.

In embodiments, the compound has the formula:

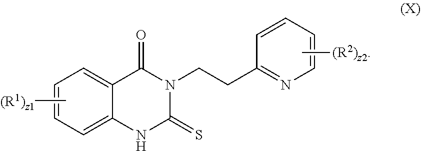

R¹, z1, R2, and z2 are as described herein, including in embodiments.

In embodiments, the compound has the formula:

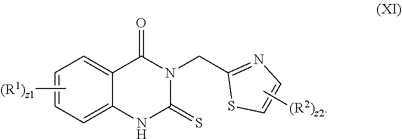

R¹, z1, R², and z2 are as described herein, including in embodiments.

In embodiments, the compound has the formula:

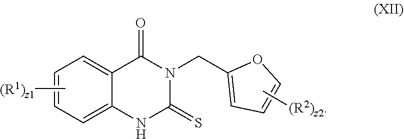

R¹, z1, R², and z2 are as described herein, including in embodiments.

In embodiments, the compound has the formula:

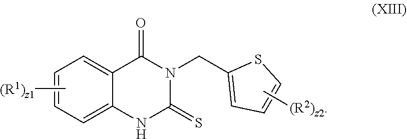

R¹, z1, R², and z2 are as described herein, including in embodiments.

In embodiments, the compound has the formula:

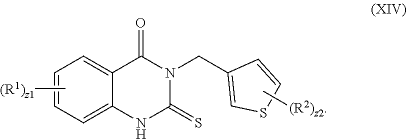

R¹, z1, R², and z2 are as described herein, including in embodiments.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B}substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 6 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy.

In embodiments, z1 is 0. In embodiments, z1 is 1. In embodiments, z1 is 2. In embodiments, z1 is 3. In embodiments, z1 is 4.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —OR^{2D}, or unsubstituted C₁-C₄ alkyl. In embodiments, R² is independently —Cl, —OCH₃, or unsubstituted methyl. In embodiments, R² is independently oxo. In embodiments, R² is independently halogen. In embodiments, R² is independently —F. In embodiments, R² is independently —Cl. In embodiments, R² is independently —Br. In embodiments, R² is independently —I. In embodiments, R² is independently —OR^{2D}. In embodiments, R² is independently —OH. In embodiments, R² is independently —OCH₃. In embodiments, R² is independently unsubstituted C₁-C₄ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted isobutyl. In embodiments, R² is independently unsubstituted tert-butyl. In embodiments, R² is independently unsubstituted 2 to 6 membered heteroalkyl. In embodiments, R² is independently unsubstituted methoxy. In embodiments, R² is independently unsubstituted ethoxy. In embodiments, R² is independently unsubstituted propoxy. In embodiments, R² is independently unsubstituted n-propoxy. In embodiments, R² is independently unsubstituted isopropoxy. In embodiments, R² is independently unsubstituted butoxy.

In embodiments, R^{2D} is independently hydrogen. In embodiments, R^{2D} is independently unsubstituted C₁-C₄ alkyl. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted n-propyl. In embodiments, R^{2D} is independently unsubstituted isopropyl. In embodiments, R^{2D} is independently unsubstituted butyl. In embodiments, R^{2D} is independently unsubstituted n-butyl. In embodiments, R^{2D} is independently unsubstituted isobutyl. In embodiments, R^{2D} is independently unsubstituted tert-butyl.

In embodiments, z2 is 0. In embodiments, z2 is 1. In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z2 is 9. In embodiments, z2 is 10. In embodiments, z2 is 11.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1} R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2}substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2} and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, the compound has the formula:

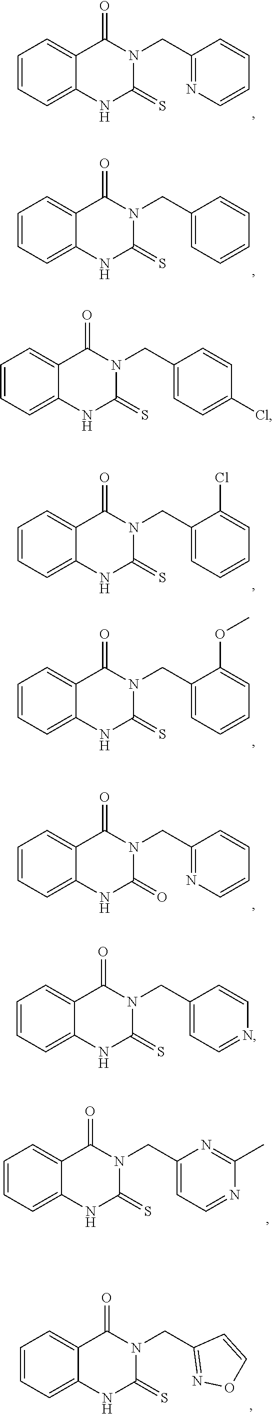

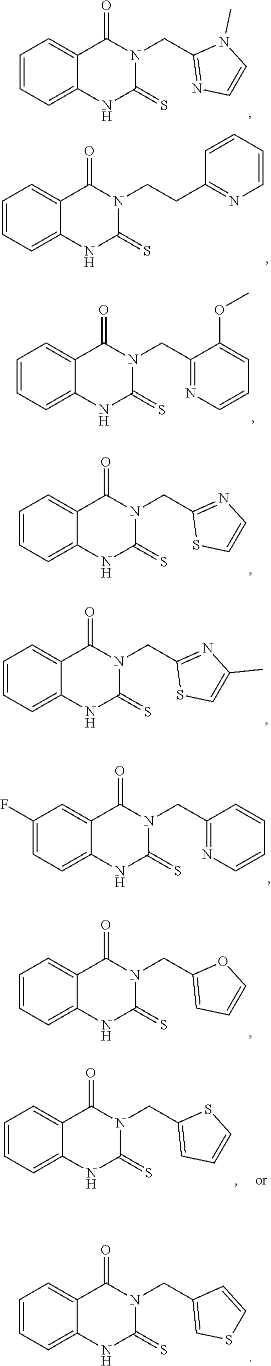

In embodiments, the compound does not have the formula:

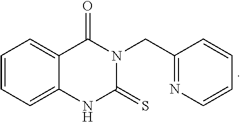 [SMILES: O=c1c2ccccc2[nH]c(=S)n1Cc1ccccn1]

In embodiments, the compound has the formula:

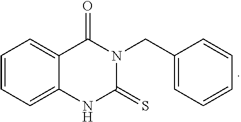 [SMILES: O=c1c2ccccc2[nH]c(=S)n1Cc1ccccc1]

In embodiments, the compound has the formula:

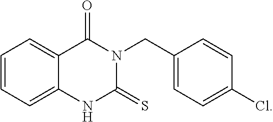 [SMILES: O=c1c2ccccc2[nH]c(=S)n1Cc1ccc(Cl)cc1]

In embodiments, the compound has the formula:

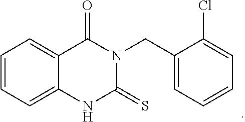 [SMILES: O=c1c2ccccc2[nH]c(=S)n1Cc1ccccc1Cl]

In embodiments, the compound has the formula:

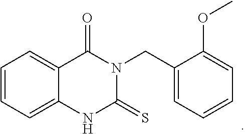 [SMILES: COc1ccccc1Cn1c(=S)[nH]c2ccccc2c1=O]

In embodiments, the compound has the formula:

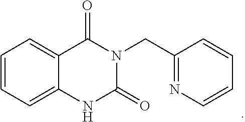 [SMILES: O=c1[nH]c2ccccc2c(=O)n1Cc1ccccn1]

In embodiments, the compound has the formula:

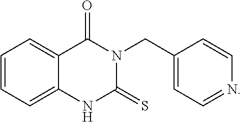 [SMILES: O=c1c2ccccc2[nH]c(=S)n1Cc1ccncc1]

In embodiments, the compound has the formula:

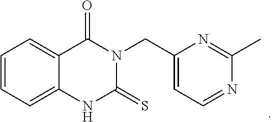 [SMILES: Cc1nccc(Cn2c(=S)[nH]c3ccccc3c2=O)n1]

In embodiments, the compound has the formula:

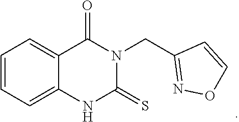 [SMILES: O=c1c2ccccc2[nH]c(=S)n1Cc1ccon1]

In embodiments, the compound has the formula:

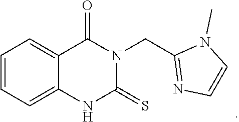 [SMILES: Cn1ccnc1Cn1c(=S)[nH]c2ccccc2c1=O]

In embodiments, the compound has the formula:

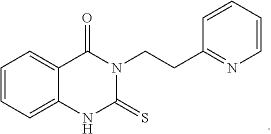 [SMILES: O=c1c2ccccc2[nH]c(=S)n1CCc1ccccn1]

In embodiments, the compound has the formula:

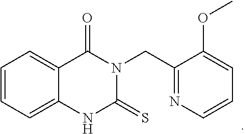 [SMILES: COc1cccnc1Cn1c(=S)[nH]c2ccccc2c1=O]

In embodiments, the compound has the formula:

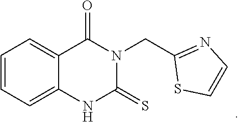 [SMILES: O=c1c2ccccc2[nH]c(=S)n1Cc1nccs1]

In embodiments, the compound has the formula:

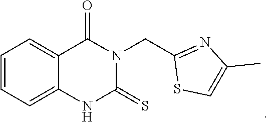 [SMILES: Cc1csc(Cn2c(=S)[nH]c3ccccc3c2=O)n1]

In embodiments, the compound has the formula:

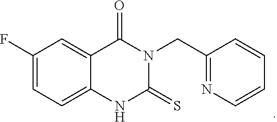 [SMILES: O=c1c2cc(F)ccc2[nH]c(=S)n1Cc1ccccn1]

In embodiments, the compound has the formula:

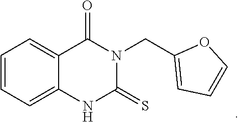 [SMILES: O=c1c2ccccc2[nH]c(=S)n1Cc1ccco1]

In embodiments, the compound has the formula:

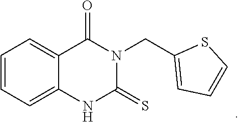 [SMILES: O=c1c2ccccc2[nH]c(=S)n1Cc1cccs1]

In embodiments, the compound has the formula:

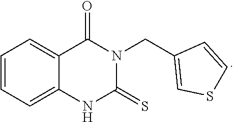 [SMILES: O=c1c2ccccc2[nH]c(=S)n1Cc1ccsc1]

In embodiments, R¹ is not halogen. In embodiments, R¹ is not —F. In embodiments, R¹ is not —Cl. In embodiments, R¹ is not —Br. In embodiments, R¹ is not —I. In embodiments, R¹ is not —C(O)NR^{1A}R^{1B}, wherein R^{1A} is substituted alkyl and R^{1B} is hydrogen. In embodiments, R¹ is not —OR^{1D}, wherein R^{1D} is hydrogen or unsubstituted C₁-C₄ alkyl. In embodiments, R is not —OH. In embodiments, R¹ is not —OCH₃. In embodiments, R¹ is not —C(O)OH. In embodiments, R is not substituted or unsubstituted C₁-C₄ alkyl. In embodiments, R is not substituted methyl. In embodiments, R is not unsubstituted methyl. In embodiments, R is not unsubstituted ethyl. In embodiments, R¹ is not unsubstituted propyl. In embodiments, R¹ is not unsubstituted n-propyl. In embodiments, R¹ is not unsubstituted isopropyl. In embodiments, R is not unsubstituted butyl. In embodiments, R is not unsubstituted n-butyl. In embodiments, R is not unsubstituted isobutyl. In embodiments, R is not unsubstituted tert-butyl. In embodiments, R¹ is not unsubstituted 5 to 6 membered heteroaryl. In embodiments, R¹ is not unsubstituted oxazolyl.

In embodiments, R² is not oxo. In embodiments, R² is not halogen. In embodiments, R² is not —F. In embodiments, R² is not —Cl. In embodiments, R² is not —Br. In embodiments, R² is not —I. In embodiments, R² is not —CF₃. In embodiments, R² is not —CN. In embodiments, R² is not —OR^{2D}. In embodiments, R² is not —OR^{2D}, wherein R^{2D} is hydrogen or unsubstituted C₁-C₄ alkyl. In embodiments, R² is not —OH. In embodiments, R² is not —OCH₃. In embodiments, R² is not —SR^{2D}, wherein R^{2D} is hydrogen or unsubstituted C₁-C₄ alkyl. In embodiments, R² is not —SCH₃. In embodiments, R² is not —NR^{2A}R^{2B}wherein R^{2A} and R^{2B} are independently hydrogen or unsubstituted C₁-C₄ alkyl. In embodiments, R² is not —N(CH₃)₃. In embodiments, R² is not —C(O)OH. In embodiments, R² is not —S(O)₂NH₂. In embodiments, R² is not substituted or unsubstituted C₁-C₄ alkyl. In embodiments, R² is not substituted methyl. In embodiments, R² is not unsubstituted methyl. In embodiments, R² is not unsubstituted ethyl. In embodiments, R² is not unsubstituted propyl. In embodiments, R² is not unsubstituted n-propyl. In embodiments, R² is not unsubstituted isopropyl. In embodiments, R² is not unsubstituted butyl. In embodiments, R² is not unsubstituted n-butyl. In embodiments, R² is not unsubstituted isobutyl. In embodiments, R² is not unsubstituted tert-butyl. In embodiments, R² is not substituted or unsubstituted 2 to 6 membered heteroalkyl. In embodiments, R² is not unsubstituted quinolinyl. In embodiments, two R² substituents are not joined to form an unsubstituted cyclopentyl.

In embodiments, z1 is not 0. In embodiments, z2 is not 0.

In embodiments, the compound does not have the formula:

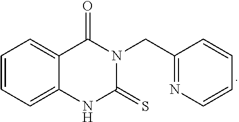 [SMILES: O=c1c2ccccc2[nH]c(=S)n1Cc1ccccn1]

In embodiments, the compound does not have the formula:

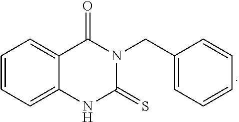 [SMILES: O=c1c2ccccc2[nH]c(=S)n1Cc1ccccc1]

In embodiments, the compound does not have the formula:

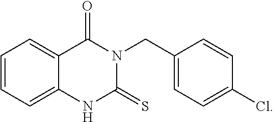 [SMILES: O=c1c2ccccc2[nH]c(=S)n1Cc1ccc(Cl)cc1]

In embodiments, the compound does not have the formula:

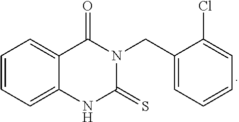 [SMILES: O=c1c2ccccc2[nH]c(=S)n1Cc1ccccc1Cl]

In embodiments, the compound does not have the formula:

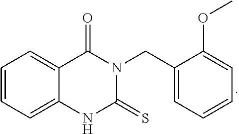 [SMILES: COc1ccccc1Cn1c(=S)[nH]c2ccccc2c1=O]

In embodiments, the compound does not have the formula:

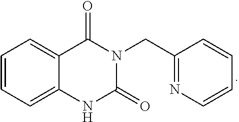 [SMILES: O=c1[nH]c2ccccc2c(=O)n1Cc1ccccn1]

In embodiments, the compound does not have the formula:

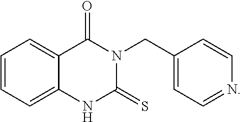 [SMILES: O=c1c2ccccc2[nH]c(=S)n1Cc1ccncc1]

In embodiments, the compound does not have the formula:

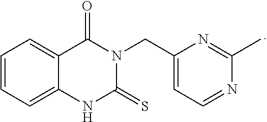 [SMILES: Cc1nccc(Cn2c(=S)[nH]c3ccccc3c2=O)n1]

In embodiments, the compound does not have the formula:

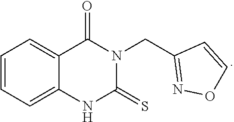 [SMILES: O=c1c2ccccc2[nH]c(=S)n1Cc1ccon1]

In embodiments, the compound does not have the formula:

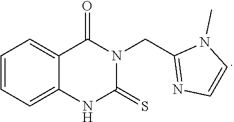 [SMILES: Cn1ccnc1Cn1c(=S)[nH]c2ccccc2c1=O]

In embodiments, the compound does not have the formula:

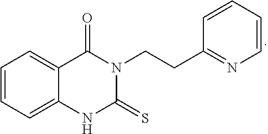 [SMILES: O=c1c2ccccc2[nH]c(=S)n1CCc1ccccn1]

In embodiments, the compound does not have the formula: H

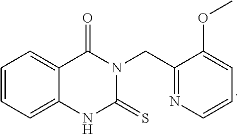 [SMILES: COc1cccnc1Cn1c(=S)[nH]c2ccccc2c1=O]

In embodiments, the compound does not have the formula:

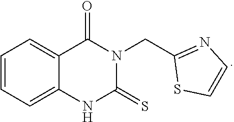 [SMILES: O=c1c2ccccc2[nH]c(=S)n1Cc1nccs1]

In embodiments, the compound does not have the formula:

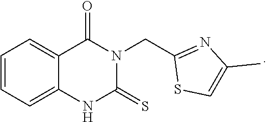 [SMILES: Cc1csc(Cn2c(=S)[nH]c3ccccc3c2=O)n1]

In embodiments, the compound does not have the formula:

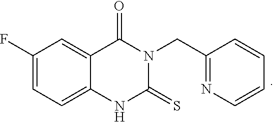 [SMILES: O=c1c2cc(F)ccc2[nH]c(=S)n1Cc1ccccn1]

In embodiments, the compound does not have the formula:

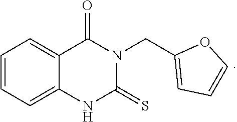 [SMILES: O=c1c2ccccc2[nH]c(=S)n1Cc1ccco1]

In embodiments, the compound does not have the formula:

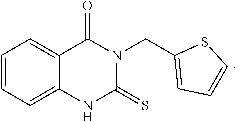 [SMILES: O=c1c2ccccc2[nH]c(=S)n1Cc1cccs1]

In embodiments, the compound does not have the formula:

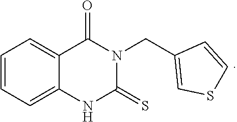 [SMILES: O=c1c2ccccc2[nH]c(=S)n1Cc1ccsc1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I), (II), (III), (IV), (V), (VI), (VII), (VIII), (IX), (X), (XI), (XII), (XIII), or (XIV).

### IV. Methods of Use

In an aspect is provided a method of treating pruritus in a subject in need thereof, the method including administering to the subject in need thereof a therapeutically effective amount of a compound described herein, or a pharmaceutically acceptable salt thereof.

In embodiments, the pruritus is pruritus associated with (e.g., caused by) psoriasis. In embodiments, the pruritus is pruritus associated with (e.g., caused by) atopic dermatitis. In embodiments, the pruritus is pruritus associated with (e.g., caused by) mastocytosis. In embodiments, the pruritus is drug-induced pruritus. In embodiments, the pruritus is pruritus associated with (e.g., caused by) urticaria.

In an aspect is provided a method of treating mast cell-mediated hypersensitivity in a subject in need thereof, the method including administering to the subject in need thereof a therapeutically effective amount of a compound described herein, or a pharmaceutically acceptable salt thereof.

In embodiments, the mast cell-mediated hypersensitivity is perioperative anaphylaxis. In embodiments, the mast cell-mediated hypersensitivity is a drug-induced allergic response.

In an aspect is provided a method of treating pain in a subject in need thereof, the method including administering to the subject in need thereof a therapeutically effective amount of a compound described herein, or a pharmaceutically acceptable salt thereof.

In embodiments, the pain is MRGPRX2-associated pain. In embodiments, the pain is inflammatory pain. In embodiments, the pain is neuropathic pain. In embodiments, the pain is post-herpetic neuralgia. In embodiments, the pain is cancer-related pain. In embodiments, the pain is peripheral neuropathy. In embodiments, the pain is inflammatory neuropathy. In embodiments, the pain is migraine.

In an aspect is provided a method of treating an inflammatory disease in a subject in need thereof, the method including administering to the subject in need thereof a therapeutically effective amount of a compound described herein, or a pharmaceutically acceptable salt thereof.

In embodiments, the inflammatory disease is dermatitis. In embodiments, the dermatitis is atopic dermatitis. In embodiments, the dermatitis is contact dermatitis. In embodiments, the dermatitis is allergic contact dermatitis. In embodiments, the inflammatory disease is urticaria. In embodiments, the urticaria is chronic urticaria. In embodiments, the urticaria is chronic spontaneous urticaria. In embodiments, the inflammatory disease is rosacea. In embodiments, the inflammatory disease is gastrointestinal disorder. In embodiments, the gastrointestinal disorder is inflammatory bowel disease. In embodiments, the gastrointestinal disorder is Crohn's disease. In embodiments, the gastrointestinal disorder is ulcerative colitis. In embodiments, the inflammatory disease is neurogenic inflammation. In embodiments, the inflammatory disease is periodontitis. In embodiments, the inflammatory disease is asthma. In embodiments, the inflammatory disease is mastocytosis. In embodiments, the inflammatory disease is atherosclerosis. In embodiments, the inflammatory disease is arthritis. In embodiments, the inflammatory disease is chronic prurigo. In embodiments, the inflammatory disease is prurigo nodularis.

In an aspect is provided a method of treating an autoimmune disease in a subject in need thereof, the method including administering to the subject in need thereof a therapeutically effective amount of a compound described herein, or a pharmaceutically acceptable salt thereof.

In embodiments, the autoimmune disease is multiple sclerosis.

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is leukemia. In embodiments, the cancer is mast cell leukemia. In embodiments, the cancer is sarcoma. In embodiments, the cancer is mast cell sarcoma. In embodiments, the cancer is skin cancer. In embodiments, the cancer is spleen cancer. In embodiments, the cancer is liver cancer. In embodiments, the cancer is intestinal cancer. In embodiments, the cancer is bone marrow cancer. In embodiments, the cancer is ovarian cancer. In embodiments, the cancer is endometrial cancer. In embodiments, the cancer is colorectal cancer. In embodiments, the cancer is breast cancer. In embodiments, the cancer is pancreatic cancer. In embodiments, the cancer is urothelial cancer.

In embodiments, the compound is a compound of formula (I), (II), (III), (IV), (V), (VI), (VII), (VIII), (IX), (X), (XI), (XII), (XIII), or (XIV).

In an aspect is provided a method of decreasing the level of activity of MRGPRX2 in a cell, said method comprising contacting the cell with an effective of a compound described herein, or a pharmaceutically acceptable salt thereof. In embodiments, the level of activity of MRGPRX2 is decreased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of MRGPRX2 is decreased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the compound binds to E164, C168, F170, G175, D176, S177, D184, W243, L247, or W248 of MRGPRX2. In embodiments, the compound binds noncovalently to E164, C168, F170, G175, D176, S177, D184, W243, L247, or W248 of MRGPRX2. In embodiments, the compound binds (e.g., noncovalently) to E164 of MRGPRX2. In embodiments, the compound binds (e.g., noncovalently) to C168 of MRGPRX2. In embodiments, the compound binds (e.g., noncovalently) to F170 of MRGPRX2. In embodiments, the compound binds (e.g., noncovalently) to G175 of MRGPRX2. In embodiments, the compound binds (e.g., noncovalently) to D176 of MRGPRX2. In embodiments, the compound binds (e.g., noncovalently) to S177 of MRGPRX2. In embodiments, the compound binds (e.g., noncovalently) to D184 of MRGPRX2. In embodiments, the compound binds (e.g., noncovalently) to W243 of MRGPRX2. In embodiments, the compound binds (e.g., noncovalently) to L247 of MRGPRX2. In embodiments, the compound binds (e.g., noncovalently) to W248 of MRGPRX2.

### V. Embodiments

Embodiment P1. A method of treating pruritus in a subject in need thereof, said method comprising administering to the subject in need thereof a therapeutically effective of a compound, or a pharmaceutically acceptable salt thereof, having the formula:

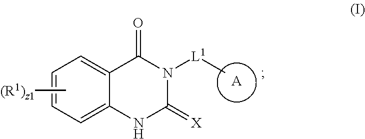

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P2. A method of treating mast cell-mediated hypersensitivity in a subject in need thereof, said method comprising administering to the subject in need thereof a therapeutically effective of a compound, or a pharmaceutically acceptable salt thereof, having the formula:

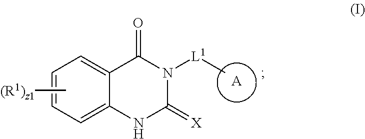

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P3. The method of embodiment P2, wherein the mast cell-mediated hypersensitivity is perioperative anaphylaxis.

Embodiment P4. The method of embodiment P2, wherein the mast cell-mediated hypersensitivity is a drug-induced allergic response.

Embodiment P5. A method of treating pain in a subject in need thereof, said method comprising administering to the subject in need thereof a therapeutically effective of a compound, or a pharmaceutically acceptable salt thereof, having the formula:

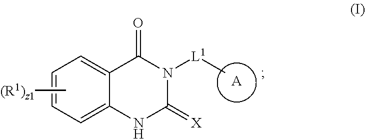

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P6. The method of embodiment P5, wherein the pain is inflammatory pain, neuropathic pain, or peripheral neuropathy.

Embodiment P7. A method of treating an inflammatory disease in a subject in need thereof, said method comprising administering to the subject in need thereof a therapeutically effective of a compound, or a pharmaceutically acceptable salt thereof, having the formula:

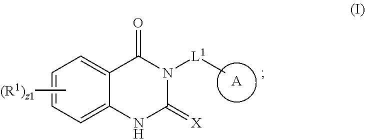

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P8. The method of embodiment P7, wherein the inflammatory disease is dermatitis, urticaria, rosacea, gastrointestinal disorder, neurogenic inflammation, periodontitis, asthma, mastocytosis, atherosclerosis, or arthritis.

Embodiment P9. The method of embodiment P8, wherein the dermatitis is atopic dermatitis.

Embodiment P10. The method of embodiment P8, wherein the dermatitis is contact dermatitis.

Embodiment P11. The method of embodiment P8, wherein the dermatitis is allergic contact dermatitis.

Embodiment P12. The method of embodiment P8, wherein the urticaria is chronic urticaria.

Embodiment P13. The method of embodiment P8, wherein the urticaria is chronic spontaneous urticaria.

Embodiment P14. The method of embodiment P8, wherein the gastrointestinal disorder is inflammatory bowel disease.

Embodiment P15. The method of embodiment P8, wherein the gastrointestinal disorder is Crohn's disease.

Embodiment P16. The method of embodiment P8, wherein the gastrointestinal disorder is ulcerative colitis.

Embodiment P17. A method of treating an autoimmune disease in a subject in need thereof, said method comprising administering to the subject in need thereof a therapeutically effective of a compound, or a pharmaceutically acceptable salt thereof, having the formula:

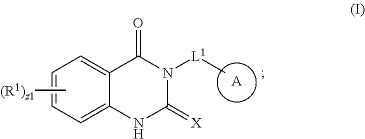

X is S or 0;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P18. The method of embodiment P17, wherein the autoimmune disease is multiple sclerosis.

Embodiment P19. A method of treating cancer in a subject in need thereof, said method comprising administering to the subject in need thereof a therapeutically effective of a compound, or a pharmaceutically acceptable salt thereof, having the formula:

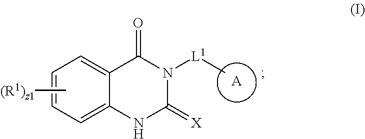

X is S or 0;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹, —CH₂X¹, —OCX¹₃, —OCH₂X¹, —OCHX¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)ORⁱc, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P20. The method of embodiment P19, wherein the cancer is leukemia, sarcoma, skin cancer, spleen cancer, liver cancer, intestinal cancer, bone marrow cancer, ovarian cancer, endometrial cancer, colorectal cancer, breast cancer, pancreatic cancer, or urothelial cancer.

Embodiment P21. The method of embodiment P19, wherein the cancer is mast cell leukemia or mast cell sarcoma.

Embodiment P22. The method of one of embodiments P1 to P21, wherein the compound has the formula:

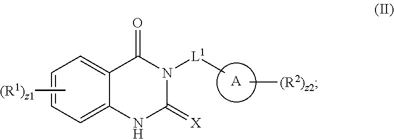

wherein
Ring A is cycloalkyl, heterocycloalkyl, aryl, or heteroaryl;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR²CNR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 11.

Embodiment P23. The method of one of embodiments P1 to P22, wherein X is S.

Embodiment P24. The method of one of embodiments P1 to P22, wherein X is 0.

Embodiment P25. The method of one of embodiments P1 to P24, wherein L¹ is unsubstituted C₁-C₄ alkylene.

Embodiment P26. The method of one of embodiments P1 to P24, wherein L¹ is unsubstituted methylene.

Embodiment P27. The method of embodiment P22, wherein Ring A is aryl or heteroaryl.

Embodiment P28. The method of embodiment P22, wherein Ring A is phenyl.

Embodiment P29. The method of embodiment P22, wherein Ring A is 5 to 6 membered heteroaryl.

Embodiment P30. The method of embodiment P22, wherein

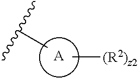

is

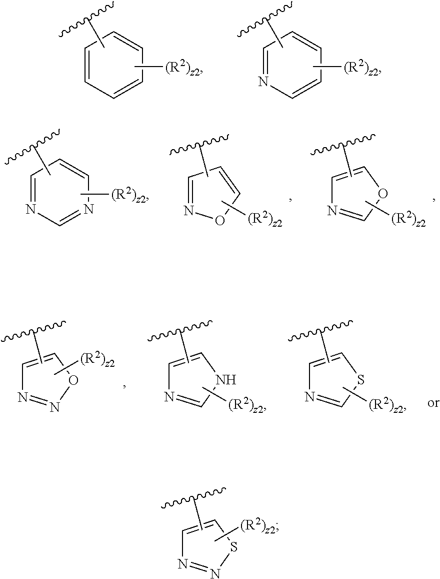

wherein z2 is an integer from 0 to 5.

Embodiment P31. The method of embodiment P30, wherein

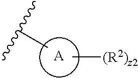

is

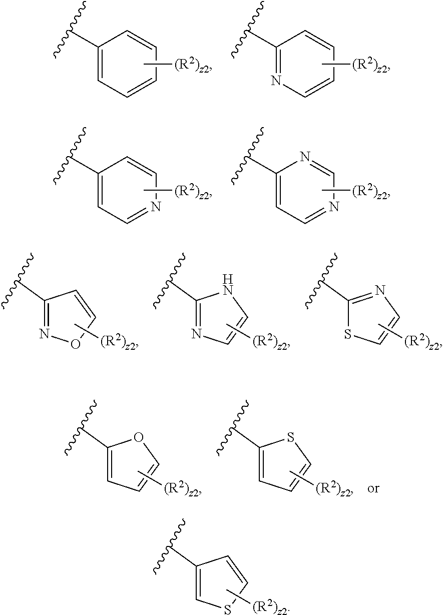

Embodiment P32. The method of one of embodiments P30 to P31, wherein z2 is 0 or 1.

Embodiment P33. The method of one of embodiments P30 to P32, wherein R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P34. The method of one of embodiments P30 to P32, wherein R² is independently halogen, —OR^{2D}, or unsubstituted C₁-C₄ alkyl.

Embodiment P35. The method of one of embodiments P30 to P32, wherein R² is independently —Cl, —OCH₃, or unsubstituted methyl.

Embodiment P36. The method of embodiment P30, wherein

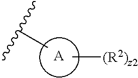

is

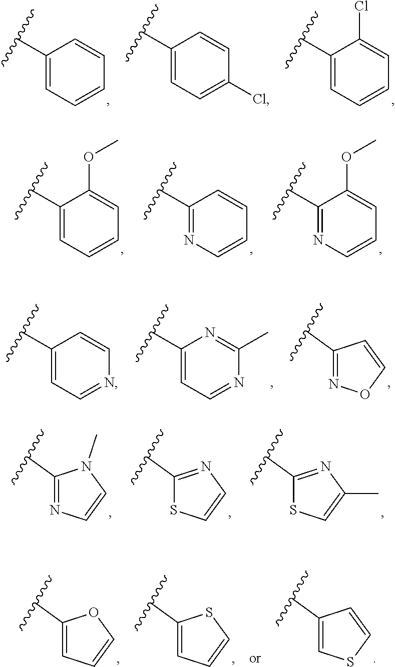

Embodiment P37. The method of embodiment P22, wherein the compound has the formula:

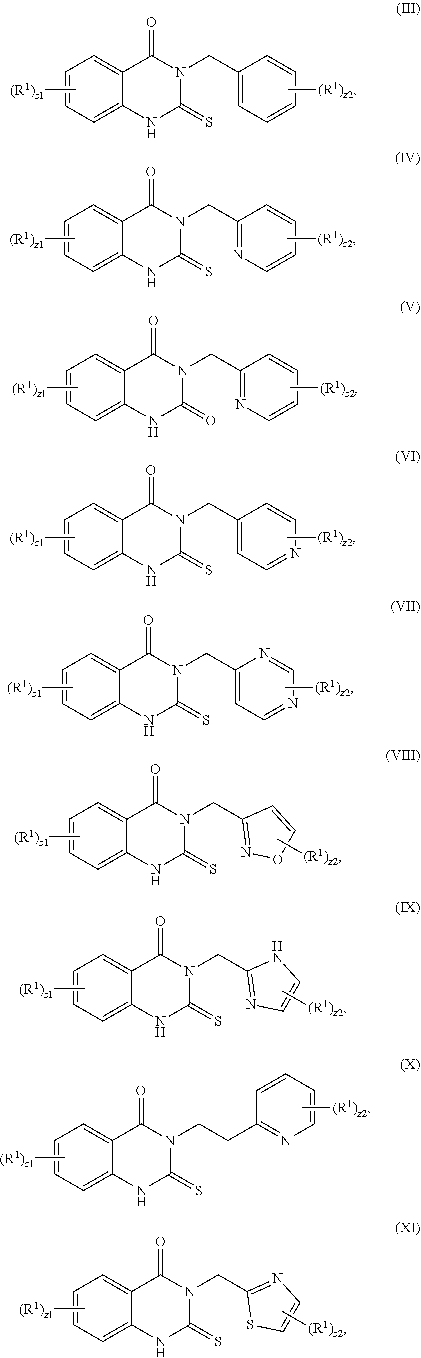

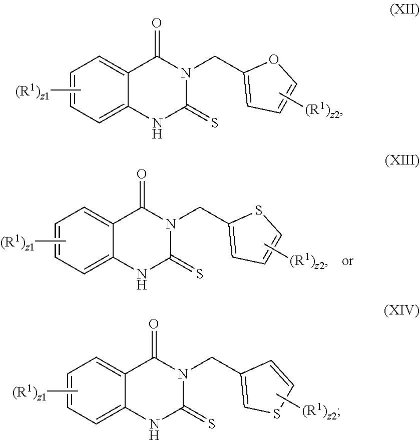

wherein z2 is an integer from 0 to 5.

Embodiment P38. The method of one of embodiments P1 to P37, wherein z1 is 0 or 1.

Embodiment P39. The method of one of embodiments P1 to P38, wherein R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P40. The method of one of embodiments P1 to P38, wherein R¹ is independently halogen.

Embodiment P41. The method of one of embodiments P1 to P38, wherein R¹ is independently —F.

Embodiment P42. The method of embodiment P1, wherein the compound is:

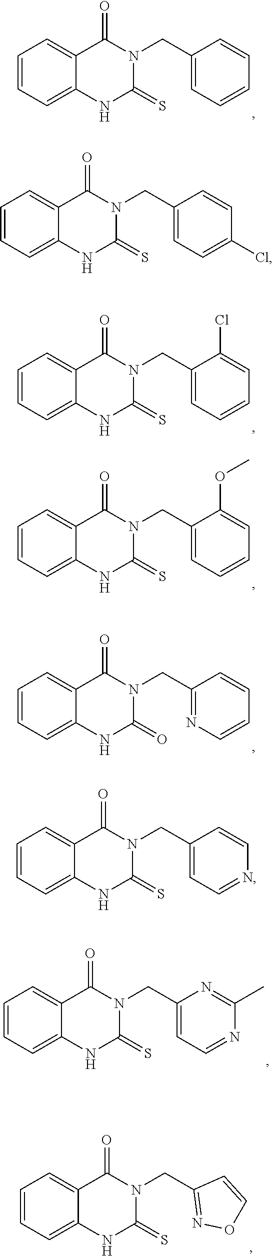

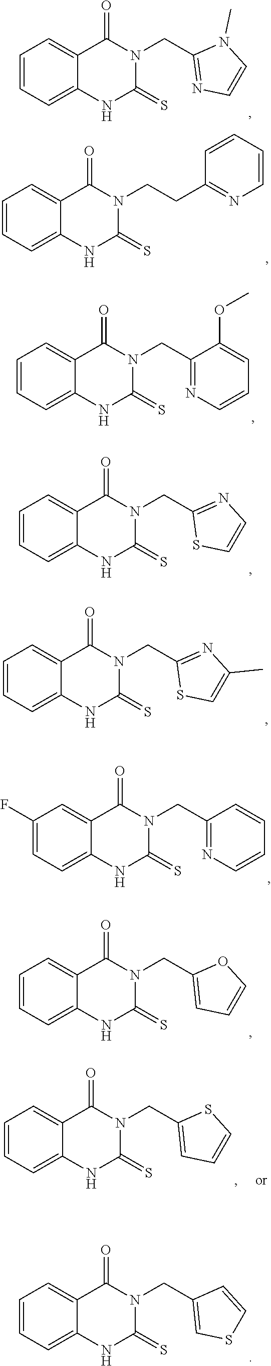

Embodiment P43. A method of decreasing the level of activity of MRGPRX2 in a cell, said method comprising contacting the cell with an effective of a compound, or a pharmaceutically acceptable salt thereof, having the formula:

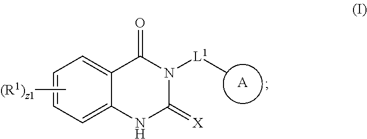

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P44. The method of embodiment P43, wherein the compound binds to E164, C168, F170, G175, D176, S177, D184, W243, L247, or W248 of MRGPRX2.

Embodiment P45. The method of embodiment P43, wherein the compound binds noncovalently to E164, C168, F170, G175, D176, S177, D184, W243, L247, or W248 of MRGPRX2.

Embodiment P46. A pharmaceutical composition comprising a compound, or a pharmaceutically acceptable salt thereof, and a pharmaceutically acceptable excipient, wherein the compound has the formula:

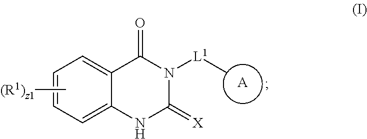

X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹, —CH₂X¹, —OCX¹₃, —OCH₂X¹, —OCHX¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Structure, Function, and Pharmacology of Human Itch GPCRs

The first Mas-related G protein coupled receptor (MRGPR) was discovered in 1986 (9) and since then a ˜40-member family of GPCRs has been identified; MRGPRs are localized to primary sensory ganglia and mast and other cells (10, 11). MRGPRs are divided into 9 major clades (viz. MRGPRA-H and MRGPRX). Of these the MRGPRX group of receptors was initially identified as a ‘primate-exclusive’ group (12) enriched in human sensory neurons. Comprising four members-MRGPRX1, X2, X3 and X4-MRGPRX4 has recently been identified to mediate cholestatic itch in humans (4) while MRGPRX2 regulates mast cell degranulation and hypersensitivity reactions (2, 13). Based on these findings, the discovery of MRGPRX-receptor selective agonists and/or antagonists have the potential to transform our understanding of this family of GPCRs and provide important therapeutic alternatives for a number of pruritic and non-pruritic disorders (14-16) an underserved therapeutic area.

We have recently identified MRGPRX2 and MRGPRX4 as targets for several commonly prescribed medications that induce itch and mast-cell mediated hypersensitivity as side effects. These drugs include the anti-diabetic agent nateglinide (17) at MRGRPX4 and several morphinan alkaloids including morphine, codeine, and dextromethorphan at MRGPRX2 (16, 18). We were able to leverage the discovery of these initial agonists in a structure-based campaign against a homology model of the MRPGRX2 receptor to discover a selective MRGPRX2 agonist (16); assays with this new reagent helped to demonstrate the specific involvement of MRGPRX2 in mast-cell degranulation. Intriguingly, MRGPRX2-mediated responses in mast cells appear to require pertussis-toxin sensitive Gαi-like proteins (9) whereas in other cells, pertussis-toxin insensitive Gαq-like G proteins (16, 19) and arrestins (16, 20) also appear to mediate signaling.

Although MRGPRX-family receptors share common sequence features among themselves, they differ from other family A GPCRs in lacking many of the canonical “trigger” motifs required for receptor activation (21). Our recent directed-evolution efforts to discover structural features responsible for activation of MRGPRX2 identified several non-canonical motifs that when mutated induce constitutive activity (21), although the mechanism(s) responsible for this remain unknown. Additionally, we showed that an African-specific variation in MRGPRX4 is strongly associated with preference for menthol cigarettes, although again how this might be achieved is unknown (15).

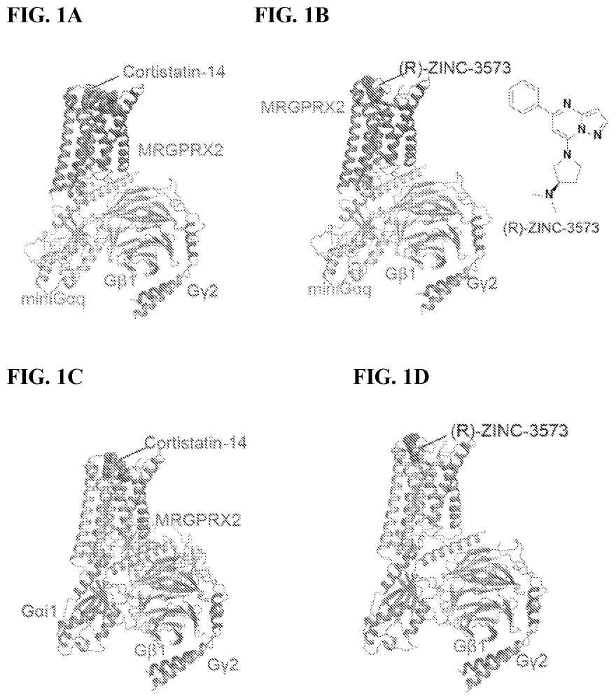

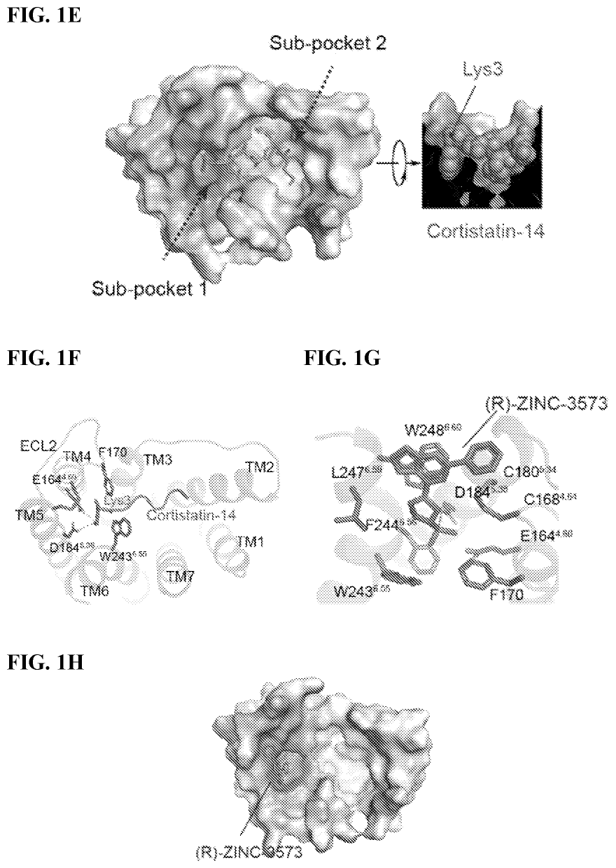

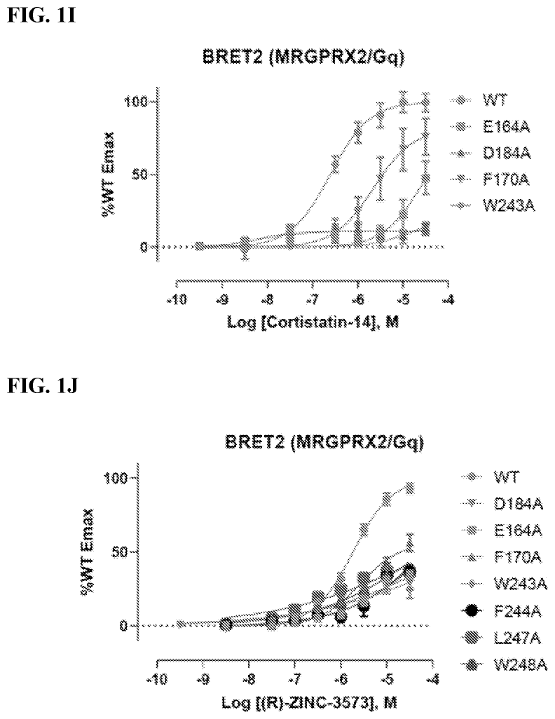

Given the importance of this receptor family and the lack of suitable chemical probes, we describe herein, inter alia, the cryo-EM structures of MRGPRX2 and MRGPRX4 in ternary complexes with G proteins and agonists (FIGS. 1A-1D). We also present the first potent and selective inverse agonist for MRGPRX2 and the first potent synthetic agonist for MRGPRX4. Taken together these results both provide templates for understanding how MRGPRX-family receptors signal and supply chemical probes useful for elucidating their functions.

MRGPRX2 couples to multiple G proteins. Several studies in transfected cells indicate preferential coupling to Gq-like G proteins (16, 19), whereas other results have shown coupling to both Gi and Gq-family G proteins (22). In mast cell lines, the involvement of pertussis-toxin-sensitive and -insensitive G proteins have been described (23, 24). We recently developed a suite of bioluminescence resonance energy transfer (BRET) reporters to reliably quantify coupling of GPCRs to individual Gα, β, and γ subunits (25) in transfected cells. Using these BRET-based reporters, we found MRGPRX2 effectively coupled to nearly all G protein families (FIG. 4). Importantly, robust coupling was determined to both Gq and Gi-family α subunits. This motivated us to determine the structures of MRGPRX2 complexed with both Gq and Gi1 using either the MRGPRX2-selective small molecule agonist (R)-ZINC-3573 (16) or the endogenous peptide agonist cortistatin-14. The structures were determined at global resolutions of 2.9 Å, 2.6 Å, 2.45 Å and 2.54 Å, respectively, via single particle cryogenic electron microscopy (cryo-EM.)

Overall structure and ligand recognition of MRGPRX2. The high-resolution maps of MRGPRX2 enabled unambiguous building of its seven transmembrane domains (7-TM) although a predicted helix 8 could not be resolved. Although some lateral motions were observed, both cortistatin-14 and (R)-ZINC-3573 adopt similar conformations in the Gi1- or Gq-coupled MRGPRX2 structures. The cryoEM maps revealed better ligand densities in the Gq-coupled MRGPRX2 complexes. Thus, our structural analysis of ligand poses is mainly focused on Gq-coupled MRGPRX2 complex structures unless otherwise specified. As we recently discussed (21), MRGPRX2 lacks, or has modified, many of the canonical trigger motifs seen in other Family A GPCRs. These include: (1) the absence of the ‘P-I-F’ (P^{5.50}, I^{3.40}, F^{6.44}) motif (26); (2) a semi-conserved DRY motif in TM3 which is ERC in MRGPR-family receptors; (3) lack of a key TM3 residue for conserved sodium binding (27); (4) and absence of the so-called ‘toggle switch’ tryptophan (W^{6.48}) (28) which is replaced by a glycine (G^{6.48}). Consequently, activated MRGPRX2 has several distinctive features in some of these key motifs. Specifically, residues of TM5 in MRGPRX2 shift downwards by two residues with respect to their canonical position, causing M196^{5.50} to move away from L117^{3.40} and F232^{6.44}. Instead, K194^{5.48} in MRGPRX2, which is in the equivalent position of P^{5.50} in other GPCRs, engages with the other two P-I-F motif residues L117^{3.40} and F232^{6.44}. Additionally, the conserved toggle switch W^{6.48} is replaced with G^{6.48} in MRGPRX2 and TM6 of MRGPRX2 is shifted towards TM3 at the extracellular side. This shift of TM6 may hinder ligand binding to the typical Family A orthosteric pocket which is frequently located above W^{6.48}. Accordingly, both corstitatin-14 and (R)-ZINC-3573 bind to MRPGRX2 at a position that is distant from the usually described Family A agonist binding site and closer to the extracellular vestibule. Finally, the conserved disulfide bond between TM3 and extracellular loop 2 (ECL2) found in Family A GPCRs is absent in MRPGX2. Instead, an inter-helix disulfide bond between C168^{4.64} and C180^{5.34}, which is predicted to be found in all the human MRGPR family receptors, was observed (FIGS. 1E-1F). Agonist induced G protein activation was greatly impaired in both C168^{4.64}A and C180^{5.34}A mutants, suggesting that this observed TM4-TM5 disulfide bond is essential for the signaling integrity of MRGPRX2.

Without the restriction introduced by the Class A canonicaTM3-ECL2 disulfide-bond, the ECL2 of MRGPRX2 is consequently flipped to the top of TM4 and TM5, resulting in an uncharacteristically wide-open extracellular ligand binding surface (FIG. 1E).

Calculation of an electrostatic potential surface revealed that the pocket of MRGPRX2 is highly negatively charged on one side (sub-pocket 1 formed by TM3-6 and ECL2) while it is relatively hydrophobic on the other side (sub-pocket 2 formed by TM1-3 and TM 6-7) (FIG. 1E, FIG. 1H).

Cortistatin-14 binds to a shallow pocket in MRGPRX2 near the extracellular loops (FIGS. 1E-1F). This likely reduces its local resolution, although we were able to model in several key cortistatin-14 residues based on the cryo-EM map. The basic residue Lys3 of cortistatin-14 binds into the negatively charged sub-pocket 1 and forms strong charge interactions with D184^{5.38} and E164^{4.60} (FIGS. 1E-1F). The remaining resolved residues of cortistatin-14 extended over W243^{6.55} and F170^{ECL.2} of MRGPRX2 and bound to the sub-pocket 2 mainly through hydrophobic interactions (FIGS. 1E-1F). This charge-hydrophobic binding mode of cortistatin-14 is consistent with prior observations showing that MRGPRX2 can bind multiple peptides that are enriched in positively charged and aromatic residues (29). Alanine substitution of W243^{6.55} completely reduced the activity of cortistatin-14 and the F170^{ECL2}A mutation also reduced the potency of cortistatin-14 by 7-fold (FIG. 1I), indicating these two bridging residues are important in maintaining a proper pocket shape for ligand engagement and receptor activation. Indeed, the diameter of sub-pocket1 is so small that only allows one side-chain of a residue to fit (FIG. 1E). Since the activation of MRGPRX2 is mainly triggered by the agonist binding to sub-pocket1, as seen in the case of (R)-ZINC-3573 (FIG. 1H), many basic peptides could potentially activate the receptor as long as a Lys or Arg side chain is fit into the acidic sub-pocket1, almost irrespective of their main-chain conformation. This may explain the long-standing puzzle why MRGPRX2 is a promiscuously activated by so many basic peptides.

Unlike cortistatin-14, the small molecule agonist (R)-ZINC-3573 bound only to the negatively charged sub-pocket1 (FIG. 1H). The N-dimethyl moiety of (R)-ZINC-3573 is directly inserted into a cavity formed by W243^{6.55}, F244^{6.56}, D184^{5.38} and E164^{4.60} where it ion-pairs with D184^{5.38} and E164^{4.60} (FIG. 1G). Consistent with these interactions, substitution of D184^{5.38} and E164^{4.60} impaired the efficacy of (R)-ZINC-3573 (FIG. 1J), and greatly reduced the potencies of peptide agonists. As many positively charged peptides and cationic small molecules activate MRGPRX2 (29, 30), ionic interactions involving these two acidic residues may be crucial for both agonist recognition and activation of MRGPRX2.

In addition to the charge interactions, (R)-ZINC-3573 also forms non-polar interactions with the receptor. The pyrazolo[1,5-a]pyrimidine moiety of (R)-ZINC-3573 lies parallel with W248^{6.60} and appears to form a π-π interaction, while its 5-phenyl moiety further extends towards ECL2 and stacks with the C168^{4.64}-C180^{5.34} disulfide (FIG. 1G). Surprisingly, (R)-ZINC-3573 has no interaction with TM3, indicating it may activate MRPGRX2 through a mechanism that is distinct from other class A GPCRs. Consistent with the pose observed in this structurestructure, most alanine mutations in the (R)-ZINC-3573 pocket greatly reduced the efficacy of (R)-ZINC-3573-stimulated Gq activation (FIG. 1J).

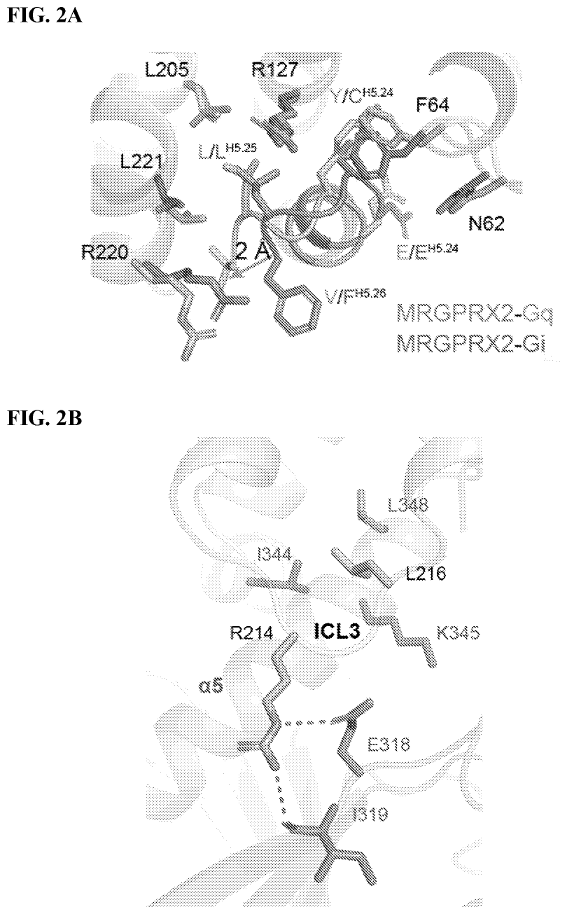

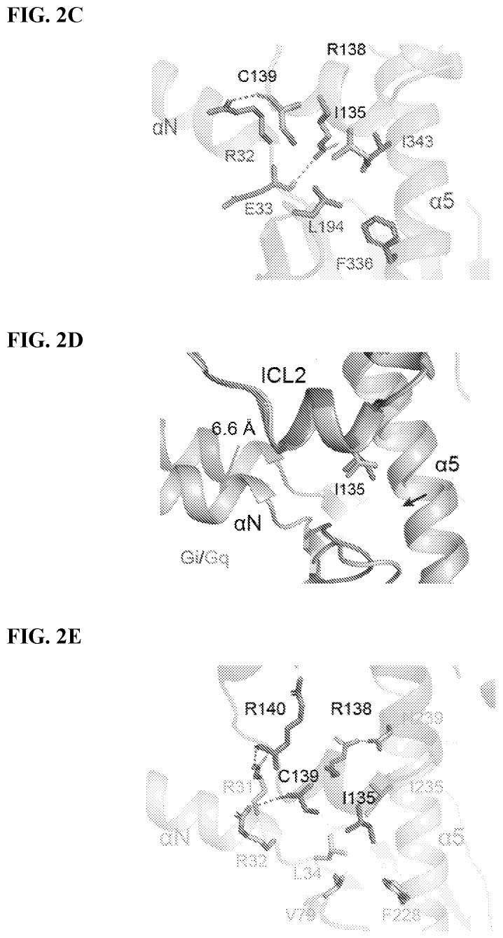

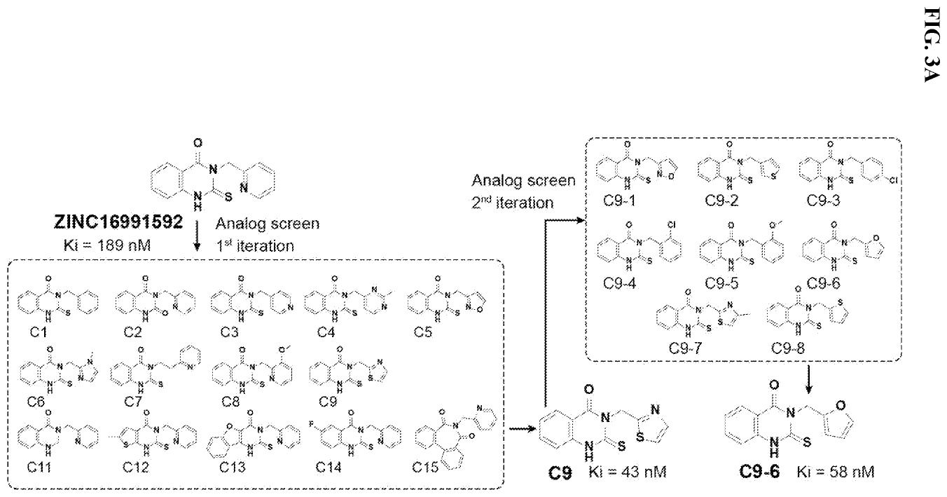

MRGPRX2 G protein coupling. MRGPRX2 displayed a similar TM6 outward movement in the Gi and Gq coupled structures. The cytoplasmic distance between TM3 and TM6 of both structures are approximately 15 A, consistent with other active-state and G-protein coupled class A GPCR structures (31-35) and distinct from inactive-state, G protein uncoupled state structures (36-38). Although the receptor structures had nearly identical main-chain conformations of the 7-TM domains, Gq and Gi1 adopt different conformations upon coupling to MRGPRX2 (FIGS. 2A-2E). The α5 helix C-terminus of Gq engages the intracellular cavity of MRGPRX2 about 2 A closer to TM6 compared to what is observed with Gi1 (FIG. 2A). This may in part be attributed to the interaction of the bulky Y243^{H5.23} of Gq with the TM2-TM3 interface of the receptor (FIG. 2A). The different engagement of Gq and Gi1 leads to a rearrangement of the surrounding cavity residues of MRGPRX2 in the two complexes (FIG. 2A). Despite these differences, both Gi1 and Gq make extensive hydrophobic and electrostatic interactions with the core though the α5 helix (FIG. 2A). A recent study showed that the common N62S variant linked to ulcerative colitis impaired G protein signaling of MRGPRX2 (20). Intriguingly, in both Gi1 and Gq-coupled structures, Asn62 is located in ICL1 and forms indirect contact with the C-terminus of the α5 helix (FIG. 2A).

Outside of the receptor core, a unique feature of the MRGPRX2-Gi1 structure is the clearly resolved intracellular loop 3 (ICL3), which formed strong interactions with Gi1 (FIG. 2B). Specifically, R214^{ICL3} extends downwards to interact with the β-sheet 6 of Gi1 and to hydrogen bond with the side chain of E318 and the carboxyl group of 1319 (FIG. 2B). In addition, L216^{ICL3} engages Gi1 α5 helix residues 1344 and L348 through non-polar interactions (FIG. 2B). Mutations of R214^{ICL3} and L216^{ICL3} impaired the efficacy of agonist stimulated Gi1 activation, suggesting ICL3 of MRGPRX2 plays a role in Gi1 coupling. By contrast, ICL3 was not resolved in Gq-coupled structure. We further noticed that agonist-stimulated Gi1 coupling of MRGPRX2 was 5-fold more potent than that of Gq (FIG. 4), which might partly be attributed to the ICL3-Gi1 interaction.

Extensive interactions of the ICL2 with the αN helix of Gi1 or Gq were also observed in both MRGPRX2 structures (FIGS. 2C-2E). In the Gi-coupled MRGPRX2, R138^{ICL2} points downwards to the C-terminus of αN helix of Gi and hydrogen-bonds with the backbone carboxyl of E33 in the αN-β1 junction (FIG. 2C). In addition, the carboxyl group of C139^{ICL2} introduces an extra hydrogen bond with R³² of Gi1 (FIG. 2C). In the Gq-coupled structure, the αN helix of Gq shifts 6.6 A outwards compared to that of Gi and interacts with the ICL2 through a distinct hydrogen bond network (FIGS. 2D-2E). Specifically, R138^{ICL2} of the Gq-coupled receptor extends towards the α5 helix of Gq and no longer interacts with the αN-β1 junction as seen in MRGPRX2-Gi1 structure. Instead, two Gq specific hydrogen bonds between the mainchain carboxyl groups of ICL2 and R³¹ and R³² of Gq were observed (FIG. 2E). In both Gq and Gi1-coupled structures, I135^{ICL2} is buried in a hydrophobic groove mainly formed by the αN-1 junction and the α5 helix of G protein (FIG. 2C, FIG. 2E). A previous study reported that this hydrophobic interaction is crucial for Gs and Gq signaling, but not for Gi1 (39). In our mutagenesis experiments, an I135^{ICL2} A substitution impaired agonist-stimulated G protein activation for both Gq and Gi, further highlighting the importance of I135^{ICL2} in G protein activation. To our knowledge this set of structures provides the first examination of a single GPCR which can couple to both Gi and Gq-family receptors.

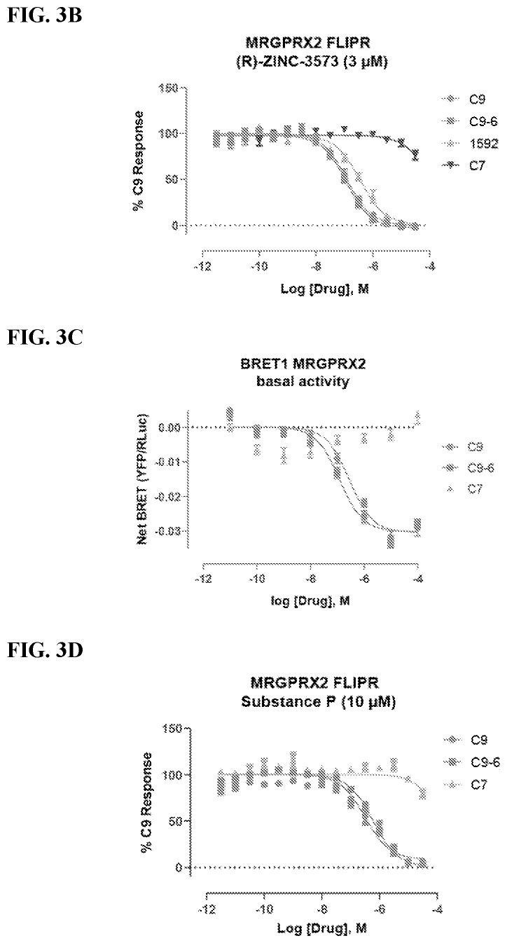

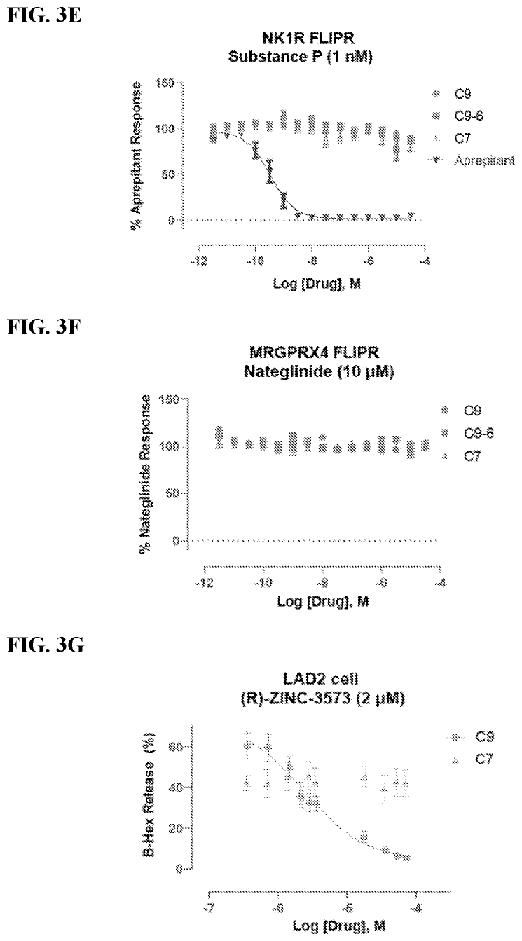

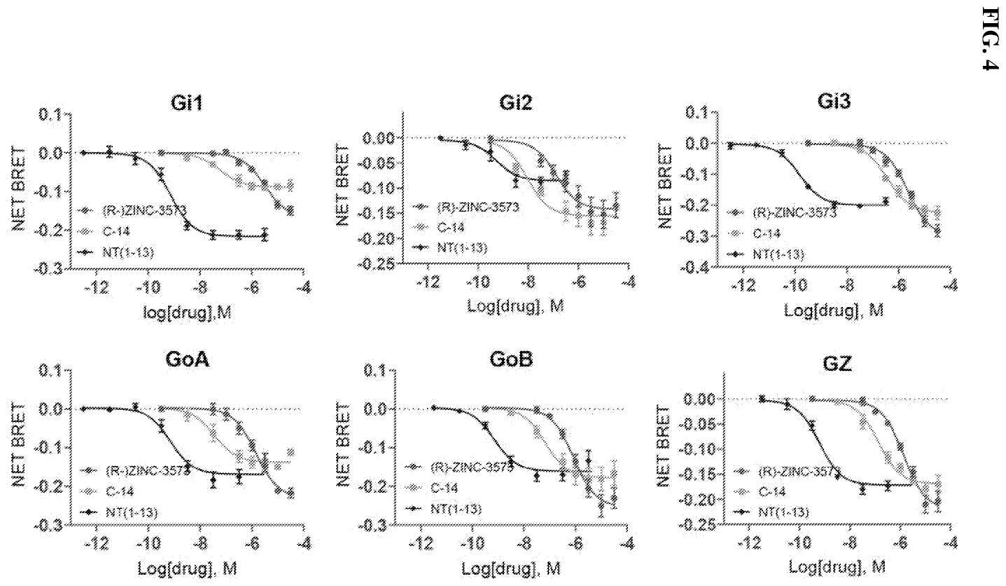

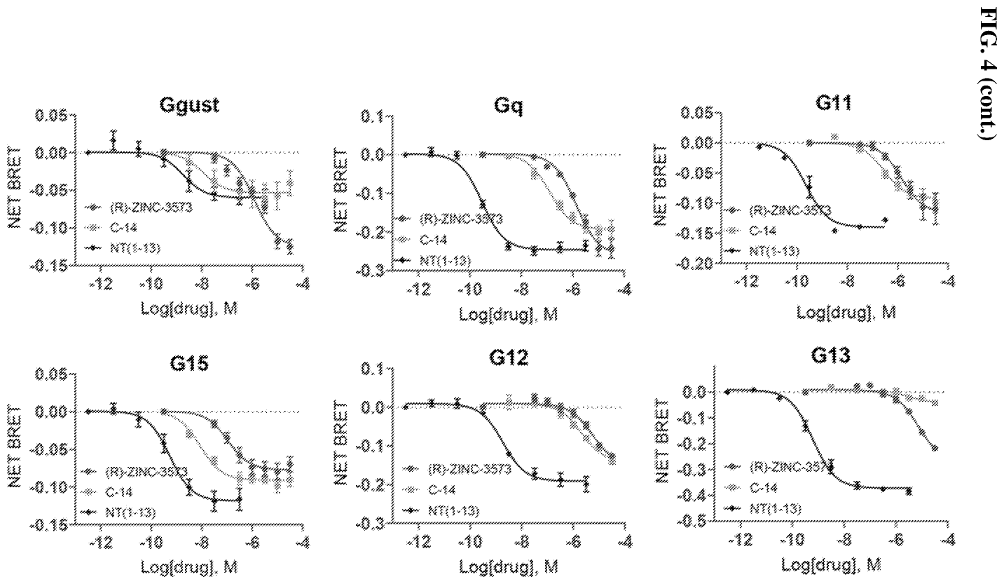

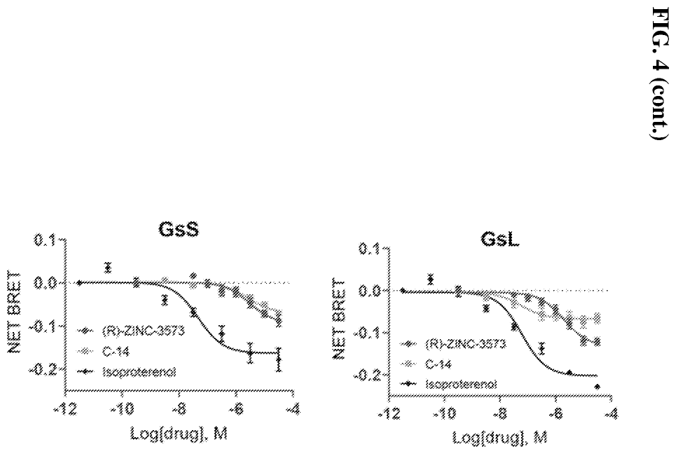

Discovery of potent MRGPRX2 antagonists. Activation of MRPGRX2 by endogenous peptides and small molecules, including many of FDA-approved drugs, may cause pseudoallergic drug reactions by inducing mast cell degranulation (29, 40). Thus, the development of a selective MRGPRX2 antagonist has substantial therapeutic potential, and may act as a chemical probe to explore the function of the receptor. To our knowledge only one prior study has identified potential MRGPRX2 antagonists, albeit of unknown selectivity (41). We confirmed the antagonist activity of the reported Compound 2 (41) (ZINC16991592, '1592) at MRGPRX2 showing that '1592 had a Ki value of 189 nM (FIG. 3A). We then searched the ZINC database (http://zinc15.docking.org) to identify potential analogues of '1592 and explore structure-activity relationships (SAR). From two rounds of analog modeling in the ultra-large make-on-demand libraries (FIG. 3A) we were able to optimize the lead compound to compounds C9-6 and C9, with Ki values of 58 and 43 nM, respectively (FIG. 3B). We found that both C9-6 and C9 are inverse agonists for MRGPRX2, inhibiting its basal activity (FIG. 3C). A recent study suggested that substance P induced inflammation and pain is at least partially mediated by MRGPRX2 rather than its canonical receptor neurokinin-1 receptor (NK1R) (3). We demonstrated both C9-6 and C9 could inhibit MRGPRX2 activation stimulated by various endogenous peptides, including substance P (FIG. 3D). Off-target profiling revealed no significant activity at a number of GPCRs, ion channels and transporters when screened at 10 μM. As well, C9-6 and C9 display no antagonist activity towards both NK1R (FIG. 3E) and the MRGPRX2 closely related receptor MRGPRX4 (FIG. 3F), revealing them as potent and selective MRGPRX2 inverse agonists potentially useful for studying the inflammatory effects of substance P and that of other MRGPRX2 agonists. Compound C9 was further tested in mast cell degranulation assay and was demonstrated to inhibit (R)-ZINC-3573-stimulated LAD2 human mast cell degranulation in a concentration-dependent manner (FIG. 3G). The potency and selectivity of C9 and C9-6 make them useful as chemical probes to further explore the biology of MRGPRX2.

Discovery of a potent MRGPRX4 agonist. MRGPRX family receptors share a high overall sequence similarity with each other, but they can differ in the broad orthosteric site. While the two acidic residues critical for the cationic agonist recognition in MRGPRX2 are conserved in MRGPRX family receptors, the other three MRGPRX receptors, including MRGPRX4, do not seem to respond to cationic agonists. To explain the potential structural basis underlying this, we determined the MRGPRX4 structure. We previously identified the antidiabetic drug nateglinide as a low micromolar agonist for MRGPRX4 (17) and used it to demonstrate MRGPRX4 is a Gq-coupling receptor. To provide a more potent agonist suitable for structural studies, we further screened MRGPRX4 in the PRESTO-Tango assay at an additional 5,000 unique small molecules and identified the structurally-related potassium channel antagonist mitiglinide as potential agonist for MRGPRX4. Subsequent concentration-response study of mitiglinide showed a similar potency as nateglinide. Both nateglinide and mitiglinide contains a D-phenylalanine base, suggesting a preliminary structure activity relationship for further refinement.

The glinides are known anti-diabetic drugs with that modulate Kir6.2/SUR1 potassium channels much more potently than they do the MRGPRX4 receptor. Thus, we sought to develop the first novel and potent MRGPRX4 ligands devoid of significant Kir6.2/SUR1 potassium channel activity; such molecules would be useful as selective probes to interrogate MRGPRX4's biological function. Upon reviewing the initial medicinal chemistry studies of nateglinide (42), a nateglinide analogue (referred as X4-1) that was reported to be inactive at the potassium channel was synthesized. Compound X4-1, which altered nateglinide's cyclohexyl to a cycloheptyl, did not substantially alter the efficacy or potency when compared to nateglinide at MRGRPX4. Our prior work on the related receptor MRGPRX2 demonstrated a preference for D-enantiomer scaffolds over L-enantiomer scaffolds (16), prompting us to synthesize X4-1 analogues with reverse stereochemistry (L) (X4-2). Unexpectedly, X4-2 showed no agonist activity at MRGPRX4, indicating MRGPRX4 prefers D-enantiomers of this scaffold. Further modifications by the addition of hydrophobic ethyl substituents to the cycloheptyl group of X4-1, namely X4-3, slightly increased MRGPRX4 activity.

To identify MRGPRX4 compounds with higher potency, we searched the ZINC database (zinc15.docking.org) (43) for commercially available compounds that were similar to X4-1 and prioritized those with larger, bulky and hydrophobic substituents. Among the identified and tested compounds, the adamantyl-substituted ZINC000004205295 ('5295) (X4-4), showed high potency at MRGRX4 with 8-fold improved potency compared to X4-1 (EC₅₀=580 nM). To further increase the compound potency, six X4-4 analogues were synthesized and tested at MRGPRX4. Of these analogues, compound MS47134, which has 3,5-dimethylation of the adamantyl group of '5295, further improved ligand potency (EC₅₀=150 nM). Compared to the original compound nateglinide, MS47134 has a 47-fold improved selectivity for MRGPRX4 over the Kir6.2/SUR1 potassium channel. Off-target profiling of MS47134 at 320 GPCRs showed activity only at MRGPRX4 and MRGPRX1 with no appreciable agonist or antagonist activity at the other 318 tested GPCRs.

Structure of MRGPRX4-Gq-agonist complex. We used our most potent MRGPRX4 agonist, MS47134, to obtain an MRGPRX4-MS47134-mGq complex suitable for structural studies. The complex structure was determined at a resolution of 2.6 A with sufficient details to unambiguously identify most side-chains in the receptor and in the complexed G protein. The overall structure of MRPGRX4 resembles MRGPRX2 at the intracellular side, and both display a prominent active conformation for G protein coupling. However, MRGPRX4 differed greatly from MRGPRX2 at the extracellular side. Compared with MRGPRX2, the extracellular tip of TM3 in MRGPRX4 was displaced 5.2 A inward with the α-helix extending one helical turn farther into the extracellular surface. This led to an ultimate inward movement of ECL1, resulting in a more compact binding pocket for the MS47134 compound. Additionally, the last helix turn of TM4 in MRGPRX2 is a loop in MRGPRX4. This loop, along with the extracellular tip of TM4, shifted into the MRGPRX4 pocket with W158^{ECL2} inserted right between TM3 and TM6, making D177^{5.38} and E157^{4.60}, the two equivalent acidic residues that are critical for cationic agonist recognition in MRGPRX2, solvent-inaccessible, which could explain MRGPRX4's apparent insensitivity to cationic agonists. Indeed, MS47134 binds to MRGPRX4 at some distance from the canonical orthosteric binding site seen for biogenic amine receptors. For instance, the negatively charged carboxyl of MS47134 is 12 A higher in the binding pocket compared with the charged amine group of 25CN—NBOH in our recently determined 5-HT2A structure (44). Unlike the negatively charged MRGPRX2 pocket, the binding pocket of MRGPRX4 had an overall positive electrostatic potential surface which likely facilitates the binding of negatively charged ligands such as bile acids (4, 5) and the potassium channel blockers nateglinide and mitiglinide (17). MS47134 was anchored by a combination of non-polar interactions and by ion pairs with R82^{2.60} and R95^{3.35}, which interact with the carboxylate of MS47134. Consistent with our SAR studies, the 3.5-dimethyl-adamantyl group of MS47134 forms extensive non-polar interactions with surrounding residues V99^{3.29}, W158^{4.61}, I239^{6.58}, Y240^{6.59}, Y250^{7.31} and Y254^{7.35}. The phenyl moiety of MS47134 is not well-resolved in our EM map, although docking suggests it interacts with R82^{2.60}, L83^{2.61}, M102^{3.32} and M258^{7.39} according to the preferred geometry of the compound. Mutagenesis studies support the pose of MS47134, as most of the mutations surrounding MS47134 greatly affected its potency. Of note is L83^{2.61}, which is annotated as the reference sequence, is highly polymorphic and replaced by S83^{2.61} in many individuals. An L83S mutation greatly attenuated the potency of both the MS47134 and nateglinide suggesting that side-effects of MRGPRX4 mediated itch may be less common in individuals with the L83S mutation.

The interactions between MRGPRX4 and Gq are quite similar to what was observed in our MRGPRX2-Gq structure, something also supported by mutagenesis data. The only major difference is that Y243^{H5.23} of Gq adopts different side-chain conformations to interact with Y130^{ICL2} upon coupling to MRGPRX4, probably resulting from the strong interaction between V61^{2.39} of MRGPRX4 and Y243^{H5.23} of Gq. Y130^{ICL2}A mutation reduced the potency of MS47134 stimulated Gq activation in MRGPRX4 while corresponding mutation Y137^{ICL2}A in MRGPRX2 has little effect on Gq activation, suggesting the interaction between Y130^{ICL2} and Gq α5 helix residue Y243^{H5.23} is more important for Gq activation in MRGPRX4. Like MRGPRX2 and other MRG-family receptors, MRGPRX4 lacks several canonical trigger motifs seen in other Family A GPCRs. In accordance with this, both MRGPRX4 and MRPGRX2 displayed a unique structural feature with an unexpected agonist binding mode in our cryoEM structure, implying a distinct receptor activation mechanism in MRGPRX family receptors.

The cryoEM structures of MRGPRX2 and MRGPRX4 illuminate unique aspects of the signalling of the unusual MRGPRX subfamily of GPCRs, and template the discovery and mechanistic understanding of selective agonist with which their function may be probed and new therapeutics designed. From a signalling perspective, the MRGPRXs lack many of the canonical motifs associated with signalling and ligand recognition in family A GPCRs. Intriguingly, the structures reveal that MRGPRX2 and MRGPRX4 agonists bind close to the extracellular solvent, over 10 A “higher” up the transmembrane helices than the orthosteric sites of most Family A GPCRs. This in turn reflects the replacement of the conserved “toggle” residue W^{6.48} by G^{6.48} in both receptors, shifting TM6 inward toward TM3 and precluding agonist binding at the canonical site. Instead, agonists bind at the more open, solvent-accessible and shallow sites that characterize these two MRGPRX receptors, and perhaps the family. As MRGPRX2 and MRGPRX4 agonists are located far away from G^{6.48}, this residue no longer acts as a “toggle” switch to sense the ligand and initiate conformational changes required for receptor activation. This suggests a unique ligand transduction mechanism in MRGPRX family receptors, which might require the formation of MRGPR-specific TM4-TM5 disulfide bond.

The discovery of MRGPRX2 antagonists and MRGPRX4 agonists, reported here and templated and understood by the structures, provides selective chemical probes and leads toward therapeutics for these orphan receptors. MRGPRX2 and MRGPRX4, and indeed the MRGPRX clade, have been implicated in mediating itch (4, 18, 45) but also neurogenic inflammation (3), atopic dermatitis (14), ulcerative colitis (20), preference for menthol cigarettes (15), pain (46) and several mast-cell mediated responses (47). The relatively potent agonists and antagonists described here, with EC₅₀ and IC₅₀ values in the mid-nanomolar range and high selectivity against 320 other GPCRs, provide chemical probes with which to explore the biology of these receptors, while they and the structures will accelerate the search for specific medications targeting this important family of GPCRs.

Collectively, the structures and tool compounds for MRGPRX2 and MRGPRX4 will accelerate the elucidation of the biology, pharmacology, and signalling mechanism of these important and understudied receptors.

### Example 2: Experimental Methods

Generation of MRPGRX2, MRGPRX4, Gi1 heterotrimer and Gq heterotrimer construct. Human MRGPRX2 and human MRGPRX4 genes were individually cloned into a modified pFastBac1 vector which contains a hemagglutinin (HA) signal peptide followed by FLAG-tag, His10-tag, and a TEV protease site at the N terminus. To facilitate protein expression and subsequent purification, thermostabilized apocytochrome b562RIL (BRIL) and HRV3C protease site were introduced at the N-terminal of receptor. For Gq protein, we used the same mini-GaqiN heterotrimer construct as what was used for 5HT2A-Gq complex (44). For Gi1 protein, a dominant-negative human Gail and Goly2 subunits were cloned into pFastbac1 and pFastDual vector individually as previously reported (48).

Receptor-G protein complex expression. The Bac-to-Bac Baculovirus Expression System (Invitrogen) was used to generate the recombinant baculovirus for protein expression. Prior to infection, viral titers were determined by flow-cytometric analysis of cells stained with gp64-PE antibody (Expression Systems). For the MRGPRX2-Gi1 complex, MRGPRX2, DN-Gi1 and Gβ1γ2 were co-expressed by infecting Tni cells at a density of 2×10⁶ cells per ml with P1 baculovirus at multiplicity of infection (MOI) ratio of 2.5:1:1. Cell was harvested by centrifugation 48 h post infection and stored at −80° C. for future use. For the MRGPRX2-Gq and MRGPRX4-Gq complex, both MRGPRX2 and MRGPRX4 were co-expressed with mini-GaqiN heterotrimer by infecting Tni cells at a density of 2×10⁶ cells per ml with P1 baculovirus at multiplicity of infection (MOI) ratio of 3:1.5, respectively. Cells were harvested by centrifugation 48 h post infection and stored at −80° C. for future use.

scFv16 expression and purification. ScFv16 gene was cloned into a modified pFastBac1 vector, expressed and purified as previously reported (49). Briefly, supernatant containing secreted scFv15 from baculovirus-infected Sf9 insect cells was collected by centrifugation at 96 h post-infection. The pH of medium was adjusted to pH 7.8 by adding Tris powder. Chelating agents were quenched by addition of 1 mM nickel and 5 mM calcium chloride and stirring at room temperature for 1 hour. After another centrifugation, the supernatant was incubated with 1 ml His60 Ni Superflow Resin (Takara) overnight at 4° C. The resin was collected next day and washed with 20 column volumes 20 mM HEPES pH 7.5, 500 mM NaCl, 10 mM imidazole. The protein was eluted with 20 mM HEPES pH 7.5, 100 mM NaCl, 250 mM imidazole and further purified by size exclusion chromatography using a Superdex 200 16/60 column (GE healthcare). Peak fraction was collected and concentrated to 2 mg ml⁻¹ for future use.

Receptor-G protein complex purification. The cell pellet of MRGPRX2-Gq complex was thawed on ice and incubated with a buffer containing 20 mM HEPES pH 7.5, 50 mM NaCl, 1 mM MgCl₂, 2.5 units of Apyrase (NEB), proteinase inhibitor and MRGPRX2 agonist (20 μM (R)-ZINC-3573 or 5 μM Cortistatin-14) at room temperature. After 1.5 h, the cell suspension was dounce homogenized. Membrane was collected by centrifugation at 25,000 rpm for 30 min using a Ti45 rotor (Beckman) and solubilized using 40 mM HEPES pH 7.5, 100 mM NaCl, 5% (w/v) glycerol, 0.6% (w/v) LMNG, 0.06% (w/v) CHS for 5 h at 4° C. with 500 μg scFv16. The solubilized proteins in the supernatants were isolated by ultra-centrifugation at 32,000 rpm for 30 min using a Ti70 rotor, and then incubate overnight at 4° C. with TALON IMAC resin (Clontech) and 20 mM imidazole. The resin was collected next day and washed with 25 column volumes 20 mM HEPES pH 7.5, 100 mM NaCl, 30 mM imidazole, 0.010% (w/v) LMNG, 0.0010% (w/v) CHS and MRGPRX2 agonist (20 μM (R)-ZINC-3573 or 5 μM Cortistatin-14). The protein was then eluted using the same buffer supplemented with 250 mM imidazole. Eluted protein was concentrated and subjected to size-exclusion chromatography on a Superdex 200 Increase 10/300 column (GE Healthcare) that was pre-equilibrated with 20 mM HEPES pH 7.5, 100 mM NaCl, 1 μM agonist, 0.00075% (w/v) MNG, 0.00025 (w/v) GDN and 0.00075% (w/v) CHS. Peak fractions were collected and incubated with 15 μl of His-tagged PreScission protease (GeneScript) and 2 μl PNGase F (NEB) at 4° C. overnight to remove the N-terminal BRIL and potential glycosylation. The protein was concentrated and further purified next day by size-exclusion chromatography using a same buffer. Peak fractions were collected and concentrated to 5 mg ml⁻¹. To ensure a full binding of the low affinity agonists to MRGPRX2, 200 μM (R)-ZINC-3573 or 100 μM Cortistantin-14 was added to the concentrated sample and incubate at cold room for 2 h prior to grid-making. The same protocol was also used for the purification of MRPGRPX2-Gi1 complex, except that the N-terminal Bril was not removed by adding PreScission protease. The MRGPRX4-Gq complex was purified using a same protocol as MRGPRX2-Gi1 complex except 20 μM MS47134 was added throughout the purification to stabilize the complex. The protein was then concentrated to 5 mg ml⁻¹ and incubate with 200 μM MS47134 for 2 h prior to grid-making.

CryoEM data collection and 3D reconstitution. The samples (3.2 μl) were applied to glow discharged Quantifoil R1.2/1.3 Au300 holey carbon grids (Ted Pella) individually and were flash frozen in a liquid ethane/propane (40/60) mixture using a Vitrobot mark IV (FEI) set at 4° C. and 100% humidity with a blot time range from 2.5 to 5 s. Images were collected using a 200 keV Talos Artica with a Gatan K3 Summit direct electron detector at a physical pixel size of 0.91 Å. Micrograph recorded movies were automatically collected using SerialEM using a multishot array (50). Data were collected at an exposure dose rate of ˜15 electrons/pixel/second as recorded from counting mode. Images were recorded for ˜2.7 seconds in 60 subframes to give a total exposure dose of ˜50 electron per Å². Following manual inspection and curation of the micrographs, particles from each dataset were selected using Blob particle picker and initial 2D classification yielded templates for subsequent template picking. A subset of the selected particles was used as a training set for Topaz and the particles were repicked from the micrographs using Topaz (51) and subjected to 2D and/or 3D classification. The picked particle coordinates from the three sets were merged yielding a subset of unique particle that survived 2D classification (i.e., duplicates were removed with a radius of 100 pixels). All subsequent three-dimensional classification and refinement steps were performed within cryoSPARC (52, 53). Multiple rounds of multi-reference refinement resolved final stack of particles that produced a map with a resolution (by FSC using the 0.143 Å cut-off criterion) (54) after Global CTF refinement and post-processing including soft masking, B-factor sharpening in cryoSPARC and filtering by local resolution (55) to generate the post-processed sharpened map. Alternative post sharpening was performed on the two half-maps using deepEMhancer (56).

Model building and refinement. Maps from deepEMhance were used for map building, refinement and subsequent structural interpretation. The dominant-negative Gi1 trimer model was adapted from the cryoEM structure of CB2-Gi complex (PDB 6PT0) (57). Gq trimer and scFv16 model was taken from 5-HT2 Å-Gq complex (PDB 6WHA) (44). The G proteins and scFv16 were docked into the cryoEM map using Chimera (58). The receptor model of MRGPRX2 and MRGPRX4 were manually build in Coot (59), followed by several rounds of real-space refinement using Phenix (60). For cortistatin-14 of MRGPRX2-Gq complex, residues 3-8 were modelled according to the map density. For the cortistatin-14 of MRGPRX2-Gi1 complex, residues 2-6 were modelled. The binding pose of MS47134 is validated by GemSpot (61). The model statistics was validated using Molprobity (62). Structural figures were prepared by Chimera or Pymol (https://pymol.org/2/)

FLIPR Ca2+ assay. Tetracycline inducible MRGPRX2 or MRGPRX4 stable cells [Flp-In™ T-REx™-293 cell which is derived from HEK 293 cells (ATCC, CRL 1573)] were maintained in DMEM containing 10% (v/v) FBS, 100 units ml⁻¹ penicillin G, 100 μg ml⁻¹ streptomycin, 100 μg ml⁻¹ hygromycin B, and 15 μg ml⁻¹ blasticidin ¹⁶. NK1 stably expressing cell were maintained in DMEM containing 10% (v/v) FBS, 100 units ml⁻¹ penicillin G and 100 μg ml⁻¹ streptomycin. On the day of assay, cells were plated into Poly-L-Lysine (PLL) coated 384-well black clear bottom cell culture plates with DMEM buffer, which is composed of 1% (v/v) dialyzed FBS, 100 units ml⁻¹ penicillin G, 100 μg ml⁻¹ streptomycin with 1 μg ml⁻¹ tetracycline at density of 20,000 cells in 40 μl per well for overnight. For NK1 stable cells, the same media used for MRGPRX2 or MRGPRX4 cells except the tetracycline were used. Medium was removed and cells were incubated with 20 μl of calcium dye (FLIPR Calcium 4 Assay Kit; Molecular Devices) diluted in assay buffer (1×HBSS, 2.5 mM probenecid, and 20 mM HEPES, pH 7.4) for 1 hour at 37° C. and 20 min at room temperature in the dark. To measure agonist activity of receptors, drug plates were prepared with increasing concentrations of test compound at 3 times the desired final concentration using drug buffer (1×HBSS, 20 mM HEPES, 0.1% (w/v) BSA, pH 7.4). Once loaded in FLIPR (Molecular Devices), basal fluorescence was measured for 10 s, then 10 μl of test compounds were added followed by continued fluorescence measurement for an additional 120 s. When measuring antagonist activity of receptors, drug plates were prepared with increasing concentrations of test compound at 4 times the desired final concentration, added as above for their potential effects on basal levels for 120 s first, followed by a 15 min incubation before addition of 10 μl of 4× of reference agonist at final concentration corresponding to the EC₈₀. Data were normalized to % reference compound stimulation and analyzed using nonlinear regression “log(agonist) vs. response” in GraphPad Prism 9.0.

Bioluminescence resonance energy transfer assay 2 (BRET2). To measure the receptor mediated G protein activation, HEK293T cells were plating either in six-well dishes containing 350-400 k cells per well, or 10-cm dishes at approximately 2 million per dish 20-24 h prior to transfection. The cells were then transfected with a 1:1:1:1 ratio of the receptor:GurLuc8:Gβ:GγGFP DNA. Transit 2020 (Mirus biosciences) was used to complex the DNA at a ratio of 3 μL Transit/μg DNA, in OptiMEM (GIBCO) at a concentration of 10 ng DNA per μL OptiMEM. After 24 h, the cells were plated in poly-L-lysine coated 96-well white clear bottom cell culture plates in plating media (DMEM+1% dialyzed FBS) at a density of 40-50,000 cells in 200 μl per well and incubated overnight. The next day, the media was carefully aspirated and cells were washed once with 60 μl of drug buffer (1×HBSS, 20 mM HEPES, 0.1% (w/v) BSA, pH 7.4), then 60 μl drug buffer containing coelenterazine 400a (nanolight technology) at 5 μM final concentration was added to each well and incubate for 5 min. Cells were then treated with 30 μl of 3× designated drug for an additional 5 minutes. After drug incubation, plates were read in an LB940 Mithras plate reader (Berthold Technologies) with a 395 nm (RLuc8-coelenterazine 400a) and 510 nm (GFP2) emission filters, at 1 s integration times. Each plate was read four times, and measurements from the fourth read were used in all analyses. BRET ratio was computed as the ratio of the GFP2 emission to rLuc8 emission. Data were normalized to % WT stimulation with indicated reference agonist and analyzed using nonlinear regression “log(agonist) vs. response” in GraphPad Prism 9.0. Log(agonist) vs normalized response—variable slope was used for (R)-ZINC-3573 pocket mutations (D184 Å, E164 Å, W243 Å, F244 Å, L247 Å, and W248 Å).

Bioluminescence resonance energy transfer assay 1 (BRET1). To test the inverse agonist activities of MRGPRX2 compounds, BRET1 recruitment assay was performed. Human MRGPRX2 containing C-terminal Renilla luciferase (RLuc8), and Venus-tagged miniGq were co-transfected at a ratio of 1:5 using HEK293T cells. After 20-24 hours, transfected cells were plated into poly-L-lysine coated 96-well clear bottom white plate in plating media (DMEM+1% (v/v) dialyzed FBS). The next day, media was decanted and cells were washed once with 60 μl drug buffer (1×HBSS, 20 mM HEPES, 0.1% (w/v) BSA, pH 7.4), then 60 μl drug buffer containing 5 μM coelenterazine h (Promega) was added to each well and incubate for 5 min. Cells were then treated with 30 μl of 3× designated drug for an additional 5 minutes. After drug incubation, plates were read in an LB940 Mithras plate reader (Berthold Technologies) for both luminescence at 485 nm and fluorescent eYFP emission at 530 nm for 1 s per well. The ratio of eYFP/RLuc was calculated per well and the net BRET ratio was calculated and fitted using “log(inhibitor) vs. response” in GraphPad Prism 9.0 to represent the inhibitory effect of MRGPX2 compound.

Inhibition screen. Binding assays were performed by the NIMH Psychoactive Drug Screening program as described previously (PMID: 23235874). Detailed binding assay protocols are available at: https://pdspdb.unc.edu/pdspWeb/content/UNC-CH %20Protocol %20Book.pdf.

Human Mast Cell activation (Beta-hexosaminidase release) Assay. Mast cell activation was assessed by measuring extracellular release of Beta-hexosaminidase, a major component of mast cell granules. To assay human mast cell Beta-hexosaminidase release, LAD2 human mast cells were seeded at a concentration of 2*10⁵ cells per well in 96-well plates in Tyrode's buffer (100 μl). 15 minutes prior to activation, varying concentrations of either C₇ or C₉ were added to the wells to reach a total volume of 190 μl. For activation, (R)-ZINC-3573 (10 μl, 2 μM) was added to each well for 30 minutes at 37° C. Separate wells received either buffer (buffer control), 0.1% Triton X-100 (total beta-hexosaminidase control), or (R)-ZINC-3573 alone (positive control) for the same amount of time. 30 μl of the supernatant were then removed from each well and added to 10 μl of NAG substrate solution (p-nitrophenyl-N-acetyl-β-D-glucosaminide) and allowed to incubate for 1 hour at 37° C. Carbonate buffer (100 μl) was then added to each well and the absorbance of each well was immediately measured at 405 nm using a plate reader. Percent degranulation (%) was calculated as follows: ((Treatment release−buffer release)/(total release−buffer release))*100. Samples were run in quadruplicate and each compound was assayed in at least two independent experiments. Data were analyzed using GraphPad Prism 9.0.

GPCRome Screening. Screening of compounds against the PRESTO-Tango GPCRome was accomplished using previously described methods with several modifications (63). First, HTLA cells were plated in white 384-well clear bottom plates (Greiner) in DMEM (Sigma) with 1% (v/v) dialyzed FBS and 10 U per mL penicillin-streptomycin (Gibco). After 24 h, the cells were transfected using PEI (Sigma) with an in-plate adapted method (64). Briefly, 17 ng per well PRESTO-Tango GPCR DNAs were resuspended in OptiMEM (Gibco) and hybridized with PEI prior to dilution and distribution into 384-well plates and subsequent addition to cells. After overnight incubation, MS47134 at 5× final concentration (3 μM) diluted in DMEM with 1% (v/v) dialyzed FBS were added to cells without replacement of the medium for 18-20 h. On the day of assay, medium and drug solution were dumped and loaded with 20 ul per well of Bright-Glo reagent (Promega). Plates were incubated for 20 min in the dark and the luminescence was counted for cells using SpectraMax Luminescence reader. Dopamine receptor D2 serves as an assay control with 0.1 mM Quinpirole. Data were analyzed using GraphPad Prism 9.0.

Surface expression. Cell surface expression of MRGPRX2 and its mutants (or MRGPRX4 and its mutants) were measured using ELISA chemiluminescence. Briefly, 48-hour post-transfected cells plated in 384 white well plates were fixed with 20 μl per well 4% (v/v) paraformaldehyde for 10 minutes at room temperature. The cells were then washed with 40 μl/well of phosphate buffered saline (PBS) twice then incubated with 20 μl per well 5% (v/v) BSA (bovine serum albumin) in PBS for 1 hour. Cells are incubated with an anti-FLAG-horseradish peroxidase-conjugated antibody (Sigma-Aldrich, A8592) diluted 1/10,000 in 5% (v/v) BSA in PBS for 1 hr at room temperature. After washing five times with 80 μl per well of PBS, 20 μl/well Super Signal Enzyme-Linked Immunosorbent Assay Pico Substrate (Thermo Fisher, #37070) was added to well for the development of signal and the luminescence was counted using a PHERAstar FSX (BMG Labtech). The luminescence signal was analyzed in GraphPad Prism 9.0. and data are normalized to the signal of WT MRGPRX2 (or WT MRGPRX4). Up to a 50% reduction in protein expression for some mutants were observed. To determine how differences in receptor expression will affect the signalling, we transfected the cells with different amount of WT MRGPRX2 pcDNA plasmid (100, 200 and 400 ng). The resulting BRET2 curves of these transfections are nearly identical, with only modest changes of EC50 and Emax values observed, thereby demonstrating that the BRET2 assay is relatively insensitive to protein expression.

Structure activity relationship analysis for ZINC16991592 and modelling of C₉, C₉-6 in active site. Preliminary structure activity relationship (SAR) on the antagonist, ZINC16991592 demonstrate that better antagonists can be developed against MRGPRX2 with higher selectivity. For the SAR, we explored substitutions both on the pyridine ring as well as the quinazole ring. Substitutions in the pyridine ring involved moving around Nitrogen in the pyridine ring, addition of halogens at ortho, meta or para positions, replacing the pyridine ring with benzene, isoxazole, 1-methylimidazole, thiazole, tetralone, thiophene and furan. Substitutions on the quinazole ring included replacement of 2-sulfanylidene-1,3-dihydroquinazolin-4-one with 1,3 dihydroquinazoline-2,4-dione or making the quinazole ring bigger with addition of another ring. Substitution of the pyridine ring with thiazole (C₉) and furan (C₉-6) resulted in the most potent and selective antagonist against MRGPRX2. The analogs with substitutions in the quinazole ring showed minimal antagonist activity against MRGPRX2, suggesting a tight SAR around this ring. All the compounds in MRGPRX2 SAR study are purchased from Enamine.

Electrophysiology. HEK293 cells were cultured in DMEM with 4.5 g L-¹ glucose, L-glutamine, and sodium pyruvate (Mediatech) containing 10% (v/v) FBS (Axenia BioLogix) and 1% (v/v) penicillin-streptomycin, at 37° C. and with 5% CO₂. Cells were lifted with trypsin-EDTA (Life Technologies) and passaged to 6-well plates (Warner Instruments) 3-4 d before recording. Transient transfection was performed with Lipofectamine 2000 (Thermo Fisher Scientific) 2 days before recording. The plasmids of human Kir6.2 and SUR1 were the gift from Dr. Show-Ling Shyng (Oregon Health and Science University), and we fused mCherry fluorescent protein to the C-terminus of Kir6.2. The vector ratio for co-transfection of Kir6.2 to SUR1 was 1:10. Before recording, cells were lifted with trypsin-EDTA, kept in modified Tyrode's saline (140 mM NaCl, 5 mM KCl, 10 mM HEPES, 2 mM CaCl₂), 1 mM MgCl₂, 10 mM glucose, pH 7.2 ˜ 7.3 with HCl), and were used within 8 hours. For recording, an aliquot of cells was transferred to a recording chamber on a Nikon-TE2000 Inverted Scope (Nikon Instruments), and transfection was confirmed with fluorescent microscopy. The pipette solution contained: 145 mM KCl, 1 mM MgCl₂, 5 mM EGTA, 2 mM CaCl₂), 20 mM HEPES, 0.3 mM K₂-ATP and 0.3 mM K₂-ADP. Patch borosilicate pipettes (Sutter Instrument) were pulled from a Sutter P-97 puller with resistances of 2-3 MΩ. Data were acquired using a Axopatch 200B amplifier controlled by Clampex 10.2 via Digidata 1550 Å (Axon Instruments), sampled at 10 kHz, filtered at 2 kHz. Membrane capacitance was around 15 pF. Rₛ was around 5 MΩ. The membrane potential was held at −80 mV and a ramp to +80 mV (1 mV/ms) was applied every second. Bath was switched to 150 mM KCl, 10 mM HEPES, 2 mM CaCl₂), and the chemical to be tested was dissolved in it and puffed with VC3-8xP pressurized perfusion system (ALA Science). For each solution, final pH was adjusted to 7.2˜7.3 with KOH, NaOH or HCl, depending on original pH and the major ion. The osmolality of each solution was 290-310 mOsm/kg. All recordings were performed at room temperature (22-24° C.). All chemicals without notes were purchased from Sigma-Aldrich.

### Example 3: Biological Data

TABLE 1

| | | pKi | Ki |
|---|---|---|---|
| Compound ID | Structure | (calc.) | (nM) |
| ZINC16991592 | 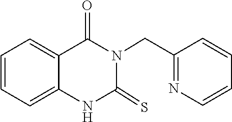 [SMILES: O=c1c2ccccc2[nH]c(=S)n1Cc1ccccn1] | 5.63 ± 0.02 | 2189 |
| 1 (ZINC98174789) | 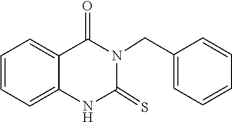 [SMILES: O=c1c2ccccc2[nH]c(=S)n1Cc1ccccc1] | 7.2 | 57 |
| 2 (ZINC5286526) | 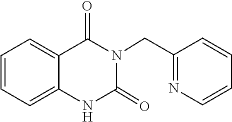 [SMILES: O=c1[nH]c2ccccc2c(=O)n1Cc1ccccn1] | 5.2 | 6176 |
| 3 (ZINC5694368) | 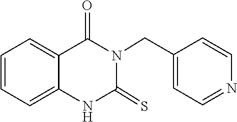 [SMILES: O=c1c2ccccc2[nH]c(=S)n1Cc1ccncc1] | n/a | n/a |
| 4 (ZINC44351689) | 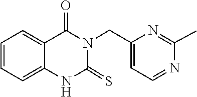 [SMILES: Cc1nccc(Cn2c(=S)[nH]c3ccccc3c2=O)n1] | n/a | n/a |
| 5 (ZINC93327152) | 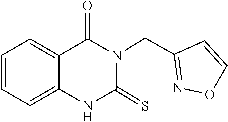 [SMILES: O=c1c2ccccc2[nH]c(=S)n1Cc1ccon1] | 6 | 895 |
| 6 (ZINC71274619) | 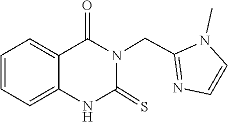 [SMILES: Cn1ccnc1Cn1c(=S)[nH]c2ccccc2c1=O] | 6.5 | 318 |
| 7 (ZINC7045547) | 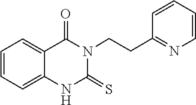 [SMILES: O=c1c2ccccc2[nH]c(=S)n1CCc1ccccn1] | 5.2 | 6519 |
| 8 (Z4655335085) | 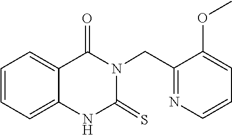 [SMILES: COc1cccnc1Cn1c(=S)[nH]c2ccccc2c1=O] | 7.2 | 57 |
| 9 (ZINC49534341) | 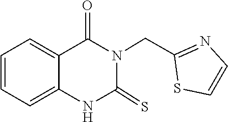 [SMILES: O=c1c2ccccc2[nH]c(=S)n1Cc1nccs1] | 7.5 | 32 |
| 14 (ZINC425284) | 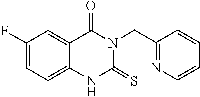 [SMILES: O=c1c2cc(F)ccc2[nH]c(=S)n1Cc1ccccn1] | 5.6 | 2517 |
| C9-1 | 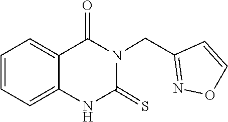 [SMILES: O=c1c2ccccc2[nH]c(=S)n1Cc1ccon1] | 6.76 ± 0.21 | 172 |
| C9-3 | 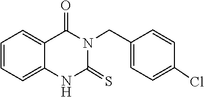 [SMILES: O=c1c2ccccc2[nH]c(=S)n1Cc1ccc(Cl)cc1] | 5.75 ± 0.16 | 1777 |
| C9-4 | 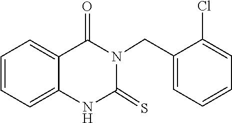 [SMILES: O=c1c2ccccc2[nH]c(=S)n1Cc1ccccc1Cl] | 5.93 ± 0.3 | 1165 |
| C9-5 | 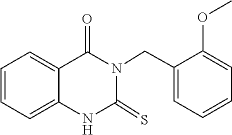 [SMILES: COc1ccccc1Cn1c(=S)[nH]c2ccccc2c1=O] | 7.01 ± 0.07 | 98 |
| C9-6 | 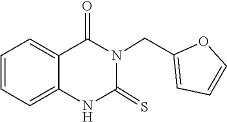 [SMILES: O=c1c2ccccc2[nH]c(=S)n1Cc1ccco1] | 7.42 ± 0.06 | 38 |
| C9-7 | 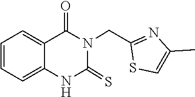 [SMILES: Cc1csc(Cn2c(=S)[nH]c3ccccc3c2=O)n1] | 6.03 ± 0.08 | 929 |
| C9-8 | 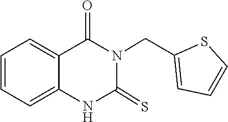 [SMILES: O=c1c2ccccc2[nH]c(=S)n1Cc1cccs1] | 6.29 ± 0.23 | 514 |
| C9-9 | 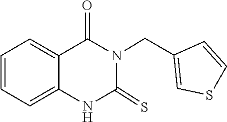 [SMILES: O=c1c2ccccc2[nH]c(=S)n1Cc1ccsc1] | 7.32 ± 0.04 | 47 |

### REFERENCES

1. Zylka, M. J., Dong, X., Southwell, A. L. & Anderson, D. J. Proceedings of the National Academy of Sciences of the United States of America 100, 10043-10048, doi:10.1073/pnas.1732949100 (2003). 2. McNeil, B. D. et al. Nature 519, 237-241, doi:10.1038/nature14022 (2015). 3. Green, D. P., Limjunyawong, N., Gour, N., Pundir, P. & Dong, X. Neuron 101, 412-420 e413, doi:10.1016/j.neuron.2019.01.012 (2019). 4. Yu, H. et al. Elife 8, doi:10.7554/eLife.48431 (2019). 5. Meixiong, J., Vasavda, C., Snyder, S. H. & Dong, X. Proceedings of the National Academy of Sciences of the United States of America 116, 10525-10530, doi:10.1073/pnas.1903316116 (2019). 6. Ikoma, A., Steinhoff, M., Stander, S., Yosipovitch, G. & Schmelz, M. Nat Rev Neurosci 7, 535-547, doi:10.1038/nm1950 (2006). 7. Greaves, M. W. & Wall, P. D. Lancet 348, 938-940, doi:10.1016/s0140-6736(96)04328-0 (1996). 8. Liu, Q. et al. Cell 139, 1353-1365, doi:10.1016/j.cell.2009.11.034 (2009). 9. Young, D., Waitches, G., Birchmeier, C., Fasano, O. & Wigler, M. Cell 45, 711-719, doi:10.1016/0092-8674(86)90785-3 (1986). 10. Dong, X., Han, S., Zylka, M. J., Simon, M. I. & Anderson, D. J. Cell 106, 619-632 (2001). 11. Solinski, H. J., Gudermann, T. & Breit, A. Pharmacol Rev 66, 570-597, doi:10.1124/pr.113.008425 (2014). 12. Lembo, P. M. et al. Nature neuroscience 5, 201-209, doi:10.1038/nn815 (2002). 13. Azimi, E. et al. JCI Insight 1, e89362, doi:10.1172/jci.insight.89362 (2016). 14. Azimi, E., Reddy, V. B. & Lemer, E. A. Brief communication: MRGPRX2, atopic dermatitis and red man syndrome. Itch (Phila) 2, doi:10.1097/itx.0000000000000005 (2017). 15. Kozlitina, J. et al. PLoS Genet 15, e1007916, doi:10.1371/joumal.pgen.1007916 (2019). 16. Lansu, K. et al. Nat Chem Biol 13, 529-536, doi:10.1038/nchembio.2334 (2017). 17. Kroeze, W. K. et al. Nature structural & molecular biology 22, 362-369, doi:10.1038/nsmb.3014 (2015). 18. Yaksh, T. L. et al. Anesthesiology 131, 132-147, doi:10.1097/ALN.0000000000002730 (2019). 19. Robas, N., Mead, E. & Fidock, M. The Journal of biological chemistry 278, 44400-44404, doi:10.1074/jbc.M302456200 (2003). 20. Chen, E. et al. Gastroenterology, doi:10.1053/j.gastro.2020.12.076 (2021). 21. English, J. G. et al. Cell 178, 748-761 e717, doi:10.1016/j.cell.2019.05.051 (2019). 22. Kamohara, M. et al. Biochem Biophys Res Commun 330, 1146-1152, doi:10.1016/j.bbrc.2005.03.088 (2005). 23. Compound Na Ayudhya, C., Roy, S., Alkanfari, I., Ganguly, A. & Ali, H. International journal of molecular sciences 20, doi:10.3390/ijms20215247 (2019). 24. Subramanian, H. et al. J Immunol 191, 345-352, doi:jimmunol.1300023 [pii] 10.4049/jimmunol.1300023 (2013). 25. Olsen, R. H. J. et al. Nat Chem Biol, doi:10.1038/s41589-020-0535-8 (2020). 26. Wacker, D. et al. Science 340, 615-619, doi:science.1232808 [pii] 10.1126/science.1232808 (2013). 27. Katritch, V. et al. Trends in biochemical sciences 39, 233-244, doi:S0968-0004(14)00048-6 [pii]10.1016/j.tibs.2014.03.002 (2014). 28. Schwartz, T. W., Frimurer, T. M., Holst, B., Rosenkilde, M. M. & Elling, C. E. Annual review of pharmacology and toxicology 46, 481-519 (2006). 29. Lu, L., Kulka, M. & Unsworth, L. D. J Leukoc Biol 102, 237-251, doi:10.1189/jlb.3RU1216-539R (2017). 30. Wolf, K. et al. J Allergy Clin Immunol, doi:10.1016/j.jaci.2020.12.655 (2021). 31. Rasmussen, S. G. et al. Nature 477, 549-555, doi:nature10361 [pii] 10.1038/nature10361 (2011). 32. Che, T. et al. Cell 172, 55-67 e15, doi:10.1016/j.cell.2017.12.011 (2018). 33. Maeda, S., Qu, Q., Robertson, M. J., Skiniotis, G. & Kobilka, B. K. Science 364, 552-557, doi:10.1126/science.aaw5188 (2019). 34. Krishna Kumar, K. et al. Cell 176, 448-458 e412, doi:10.1016/j.cell.2018.11.040 (2019). 35. Kim, K. et al. Cell 182, 1574-1588.e1519., doi:10.1016/j.cell.2020.08.024. (2020). 36. Rosenbaum, D. M. et al. Science 318, 1266-1273 (2007). 37. Cherezov, V. et al. Science 318, 1258-1265 (2007). 38. Che, T. et al. Nature communications 11, 1145, doi:10.1038/s41467-020-14889-7 (2020). 39. Moro, O., Lameh, J. & Sadee, W. The Journal of biological chemistry 268, 6862-6865 (1993). 40. Grimes, J. et al. Pharmacol Res Perspect 7, e00547, doi:10.1002/prp2.547 (2019). 41. Ogasawara, H., Furuno, M., Edamura, K. & Noguchi, M. J Leukoc Biol 106, 1069-1077, doi:10.1002/JLB.2AB1018-405R (2019). 42. Shinkai, H. et al. Journal of medicinal chemistry 32, 1436-1441, doi:10.1021/jm00127α006 (1989). 43. Irwin, J. J. & Shoichet, B. K. Journal of chemical information and modeling 45, 177-182, doi:10.1021/ci049714+(2005). 44. Kim, K. et al. Cell 182, 1574-1588.e1519., doi:10.1016/j.cell.2020.08.024. (2020). 45. Meixiong, J. et al. Elife 8, doi:10.7554/eLife.44116 (2019). 46. Li, Z. et al. Proceedings of the National Academy of Sciences of the United States of America 114, E1996-E2005, doi:10.1073/pnas.1615255114 (2017). 47. Thapaliya, M., Chompunud Na Ayudhya, C., Amponnawarat, A., Roy, S. & Ali, H. Curr Allergy Asthma Rep 21, 3, doi:10.1007/si 1882-020-00979-5 (2021). 48. Draper-Joyce, C. J. et al. Nature 558, 559-563, doi:10.1038/s41586-018-0236-6 (2018). 49. Koehl, A. et al. Nature 558, 547-552, doi:10.1038/s41586-018-0219-7 (2018). 50. Mastronarde, D. N. J Struct Biol 152, 36-51, doi:10.1016/j.jsb.2005.07.007 (2005). 51. Bepler, T., Kelley, K., Noble, A. J. & Berger, B. Nat Commun 11, 5208, doi:10.1038/s41467-020-18952-1 (2020). 52. Punjani, A., Rubinstein, J. L., Fleet, D. J. & Brubaker, M. A. Nat Methods 14, 290-296, doi:10.1038/nmeth.4169 (2017). 53. Punjani, A., Zhang, H. & Fleet, D. J. Nat Methods 17, 1214-1221, doi:10.1038/s41592-020-00990-8 (2020). 54. Rosenthal, P. B. & Henderson, R. J Mol Biol 333, 721-745, doi:10.1016/j.jmb.2003.07.013 (2003). 55. Heymann, J. B. & Belnap, D. M. J Struct Biol 157, 3-18, doi:10.1016/j.jsb.2006.06.006 (2007). 56. Sanchez-Garcia, R. et al. bioRxiv (2020). 57. Xing, C. et al. Cell 180, 645-654 e613, doi:10.1016/j.cell.2020.01.007 (2020). 58. Pettersen, E. F. et al. J Comput Chem 25, 1605-1612, doi:10.1002/jcc.20084 (2004). 59. Emsley, P. & Cowtan, K. Acta Crystallogr D Biol Crystallogr 60, 2126-2132, doi:10.1107/50907444904019158 (2004). 60. Adams, P. D. et al. Acta Crystallogr D Biol Crystallogr 66, 213-221, doi:10.1107/50907444909052925 (2010). 61. Robertson, M. J., van Zundert, G. C. P., Borrelli, K. & Skiniotis, G. Structure 28, 707-716 e703, doi:10.1016/j.str.2020.04.018 (2020). 62. Chen, V. B. et al. Acta Crystallogr D Biol Crystallogr 66, 12-21, doi:10.1107/50907444909042073 (2010). 63. Kroeze, W. K. et al. Nature structural & molecular biology 22, 362-U328, doi:10.1038/nsmb.3014 (2015). 64. Longo, P. A., Kavran, J. M., Kim, M. S. & Leahy, D. J. Methods Enzymol 529, 227-240, doi:10.1016/B978-0-12-418687-3.00018-5 (2013).

What is claimed is:

1. A method of treating pruritus in a subject in need thereof, said method comprising administering to the subject in need thereof a therapeutically effective of a compound, or a pharmaceutically acceptable salt thereof, having the formula: 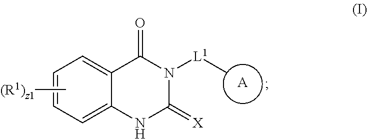
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

2. A method of treating mast cell-mediated hypersensitivity in a subject in need thereof, said method comprising administering to the subject in need thereof a therapeutically effective of a compound, or a pharmaceutically acceptable salt thereof, having the formula: 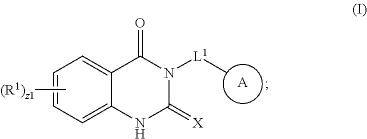
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

3. The method of claim 2, wherein the mast cell-mediated hypersensitivity is perioperative anaphylaxis.

4. The method of claim 2, wherein the mast cell-mediated hypersensitivity is a drug-induced allergic response.

5. A method of treating pain in a subject in need thereof, said method comprising administering to the subject in need thereof a therapeutically effective of a compound, or a pharmaceutically acceptable salt thereof, having the formula: 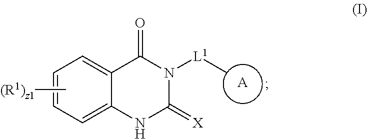
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

6. The method of claim 5, wherein the pain is inflammatory pain, neuropathic pain, peripheral neuropathy, or migraine.

7. A method of treating an inflammatory disease in a subject in need thereof, said method comprising administering to the subject in need thereof a therapeutically effective of a compound, or a pharmaceutically acceptable salt thereof, having the formula: 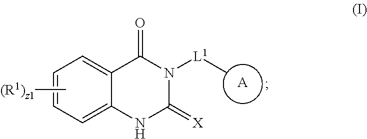
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and RD are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

8. The method of claim 7, wherein the inflammatory disease is dermatitis, urticaria, rosacea, gastrointestinal disorder, neurogenic inflammation, periodontitis, asthma, mastocytosis, atherosclerosis, arthritis, chronic prurigo, prurigo nodularis, or chronic spontaneous urticaria.

9. The method of claim 8, wherein the dermatitis is atopic dermatitis.

10. The method of claim 8, wherein the dermatitis is contact dermatitis.

11. The method of claim 8, wherein the dermatitis is allergic contact dermatitis.

12. The method of claim 8, wherein the urticaria is chronic urticaria.

13. The method of claim 8, wherein the urticaria is chronic spontaneous urticaria.

14. The method of claim 8, wherein the gastrointestinal disorder is inflammatory bowel disease.

15. The method of claim 8, wherein the gastrointestinal disorder is Crohn's disease.

16. The method of claim 8, wherein the gastrointestinal disorder is ulcerative colitis.

17. A method of treating an autoimmune disease in a subject in need thereof, said method comprising administering to the subject in need thereof a therapeutically effective of a compound, or a pharmaceutically acceptable salt thereof, having the formula: 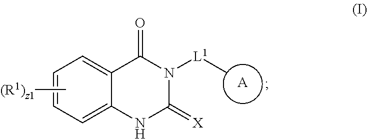
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

18. The method of claim 17, wherein the autoimmune disease is multiple sclerosis.

19. A method of treating cancer in a subject in need thereof, said method comprising administering to the subject in need thereof a therapeutically effective of a compound, or a pharmaceutically acceptable salt thereof, having the formula: 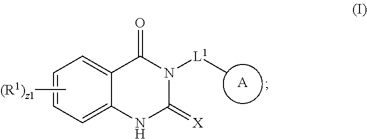
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and RD are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

20. The method of claim 19, wherein the cancer is leukemia, sarcoma, skin cancer, spleen cancer, liver cancer, intestinal cancer, bone marrow cancer, ovarian cancer, endometrial cancer, colorectal cancer, breast cancer, pancreatic cancer, or urothelial cancer.

21. The method of claim 19, wherein the cancer is mast cell leukemia or mast cell sarcoma.

22. The method of one of claims 1 to 21, wherein the compound has the formula: 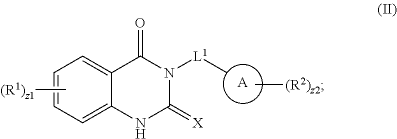
wherein
Ring A is cycloalkyl, heterocycloalkyl, aryl, or heteroaryl;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 11.

23. The method of one of claims 1 to 21, wherein X is S.

24. The method of one of claims 1 to 21, wherein X is 0.

25. The method of one of claims 1 to 21, wherein L¹ is unsubstituted C₁-C₄ alkylene.

26. The method of one of claims 1 to 21, wherein L¹ is unsubstituted methylene.

27. The method of claim 22, wherein Ring A is aryl or heteroaryl.

28. The method of claim 22, wherein Ring A is phenyl.

29. The method of claim 22, wherein Ring A is 5 to 6 membered heteroaryl.

30. The method of claim 22, wherein 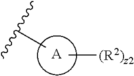
is 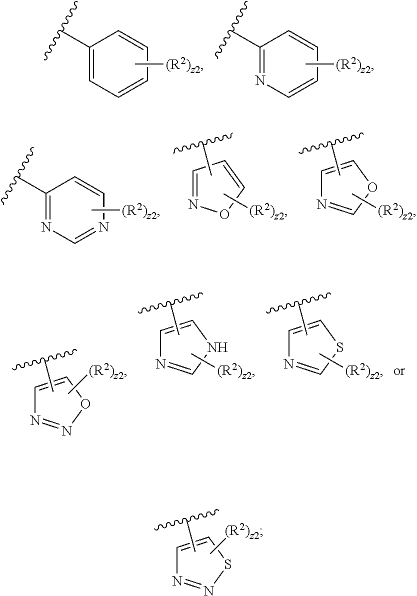
wherein
z2 is an integer from 0 to 5.

31. The method of claim 30, wherein 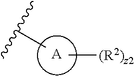
is

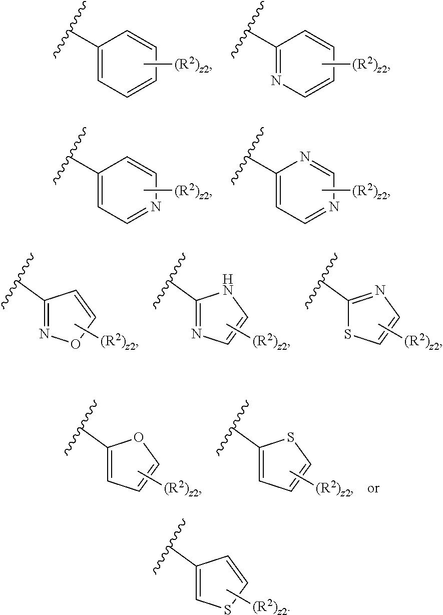

32. The method of claim 30, wherein z2 is 0 or 1.

33. The method of claim 30, wherein R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

34. The method of claim 30, wherein R² is independently halogen, —OR^{2D}, or unsubstituted C₁-C₄ alkyl.

35. The method of claim 30, wherein R² is independently —Cl, —OCH₃, or unsubstituted methyl.

36. The method of claim 30, wherein 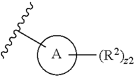
is

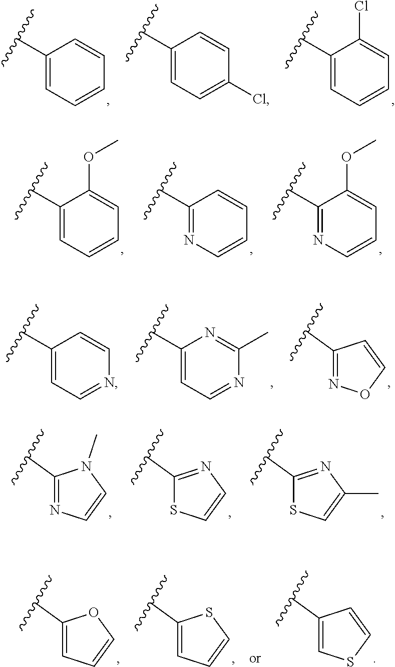

37. The method of claim 22, wherein the compound has the formula: 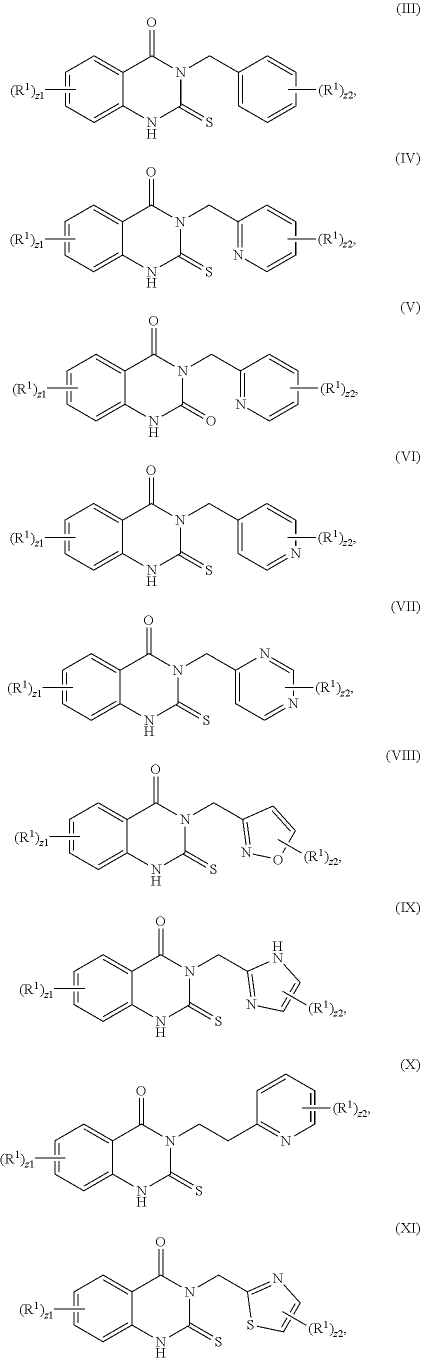 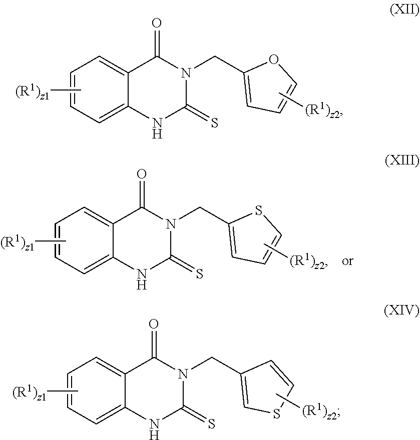
wherein
z2 is an integer from 0 to 5.

38. The method of one of claims 1 to 21, wherein z1 is 0 or 1.

39. The method of one of claims 1 to 21, wherein R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

40. The method of one of claims 1 to 21, wherein R¹ is independently halogen.

41. The method of one of claims 1 to 21, wherein R¹ is independently —F.

42. The method of claim 1, wherein the compound is:

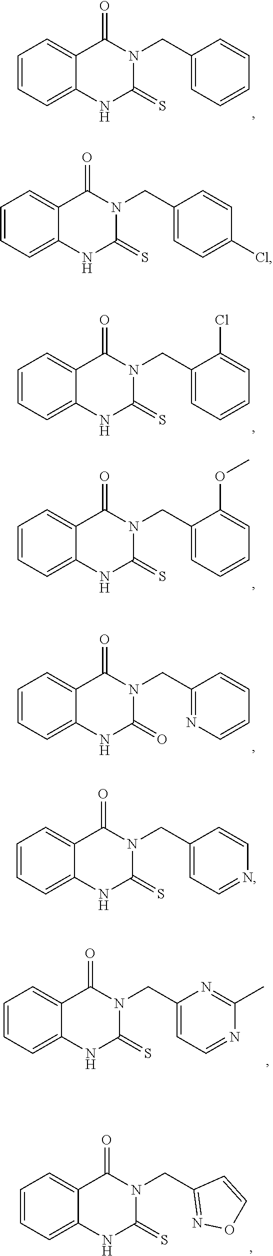

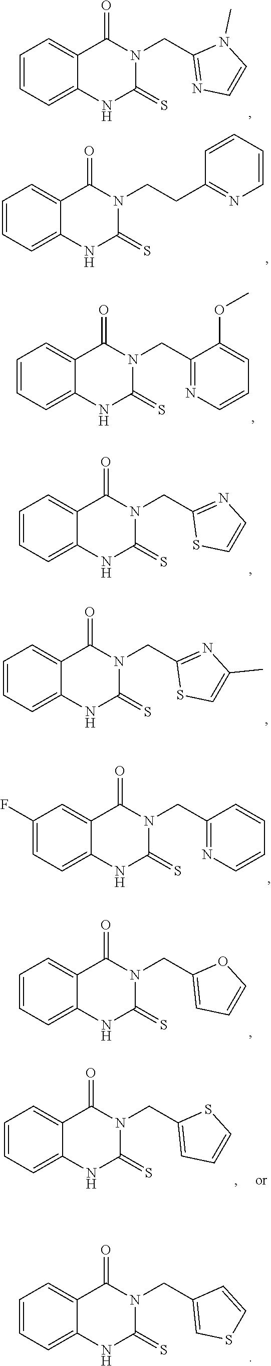

43. A method of decreasing the level of activity of MRGPRX2 in a cell, said method comprising contacting the cell with an effective of a compound, or a pharmaceutically acceptable salt thereof, having the formula: 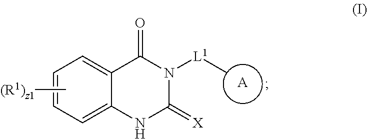
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
R¹ is independently halogen, —CX¹₃, —CHX¹, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

44. The method of claim 43, wherein the compound binds to E164, C168, F170, G175, D176, S177, D184, W243, L247, or W248 of MRGPRX2.

45. The method of claim 43, wherein the compound binds noncovalently to E164, C168, F170, G175, D176, S177, D184, W243, L247, or W248 of MRGPRX2.

46. A pharmaceutical composition comprising a compound, or a pharmaceutically acceptable salt thereof, and a pharmaceutically acceptable excipient, wherein the compound has the formula: 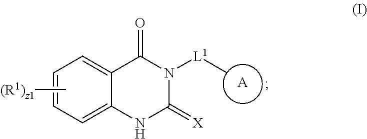
X is S or O;
Ring A is substituted or unsubstituted cycloalkyl, substituted or unsubstituted heterocycloalkyl, substituted or unsubstituted aryl, or substituted or unsubstituted heteroaryl;
L¹ is substituted or unsubstituted alkylene;
9 R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B}substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.


SEQUENCE LISTING (1 sequence)

SEQ ID NO: 1 - protein, 330 aa, synthetic construct
MDPTTPAWGTESTTVNGNDQALLLLCGKETLIPVFLILFIALVGLVGNGFVLWLLGFRMRRNAFSVYVLSLAGADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDHSEGCFRQGTPEMSRSSLV